(19) United States
(12) Patent Application Publication
(10) Pub. No.: US 2007/0220627 A1
(43) Pub. Date: Sep. 20, 2007

(54) Methods and Compositions for the Selection of a Transgenic Plant

(75) Inventors: Margit C. Ross, Johnston, IA (US); Zuo-yu Zhao, Johnston, IA (US); Dennis L. Bidney, Urbandale, IA (US); Drew L. Brumbaugh, Koloa, HI (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/559,022

(22) Filed: Nov. 13, 2006

Related U.S. Application Data
(60) Provisional application No. 60/784,098, filed on Mar. 20, 2006.

(51) Int. Cl. A01H 5/00

(52) U.S. Cl. 800/278; 800/306; 800/320; 800/320.1; 800/320.2; 800/320.3; 800/315; 800/317.3; 800/312

(57) ABSTRACT

Compositions and methods are provided to screen, identify, select, isolate, and/or regenerate targeted integration events using seed priming. Seed priming provides the identification of a seed having stably incorporated into its genome a site-specific recombinase mediated integration of a selectable marker at a target locus operably linked to a promoter active in the seed.

### CROSS-REFERENCE TO RELATED APPLICATIONS

This application claims the benefit of U.S. Application Ser. No. 60/784,098 filed Mar. 20, 2006, which is herein incorporated by reference in its entirety.

### FIELD OF THE INVENTION

The invention relates to the genetic modification of plants. In particular, methods and compositions are provided for the selection of transgenic plants.

### BACKGROUND

The frequency of integration of a polynucleotide into a desired chromosomal position is low, requiring the generation and screening of large numbers of progeny. Screening is typically time and labor intensive. Methods to improve the selection and screening process are needed. Methods to improve the efficiency of screening for targeted integration events in plants are needed. The present invention provides methods and compositions using seed priming with targeted integration systems, providing increased efficiency for screening and/or selection of targeted events.

### SUMMARY

Compositions and methods are provided to screen, identify, select, isolate, and/or regenerate targeted integration events using seed priming. Seed priming provides the identification of a seed having stably incorporated into its genome a site-specific recombinase-mediated integration of a selectable marker at a target locus operably linked to a promoter active in the seed.

### DETAILED DESCRIPTION

Compositions and methods to prime seeds include, but are not limited to the following:

1. A method to identify a plant having a DNA construct stably incorporated in its genome comprising:
(a) providing a population of seeds, wherein at least one seed in the population has stably incorporated into its genome the DNA construct comprising the following operably linked components in the following order: a promoter active in the seed, a first recombination site, a polynucleotide encoding a selection marker that confers resistance to a selective agent, and a second recombination site;
(b) contacting the population of seeds with a priming matrix comprising an effective concentration of the selection agent for a time sufficient to produce a population of primed seeds; and,
(c) incubating the population of primed seed under germination conditions, thereby identifying plants having the DNA construct stably incorporated into their genome.

2. The method of 1, wherein the first and the second recombination sites are dissimilar and non-recombinogenic with each other.

3. The method of any one of 1 or 2, wherein the selective agent comprises an herbicide, a growth regulator, or an antibiotic.

4. The method of any one of 1-3, wherein the selective agent is selected from the group consisting of glufosinate, phosphinothricin, glyphosate, bromoxynil, methotrexate, imidazolinones, sulfonylureas, cyanamide, kanamycin, G418, neomycin, and hygromycin B.

5. The method of any one of 1-3, wherein the selection marker is selected from the group consisting of Bar, phosphinothricin acetyltransferase (PAT), glyphosate oxidoreductase (GOX), 5-enolpyruvylshikimate-3-phosphate synthase (EPSPS), CP4, glyphosate N-acetyltransferase (GAT), bromoxynil nitrilase (BXN), dihydrofolate reductase, acetolactate synthase (ALS), cyanamide hydratase (CAH), neomycin phosphotransferase (nptl), aminoglycoside 3′-phosphotransferase (APH3′ II), and hygromycin phosphotransferase (hph).

6. The method of any one of 1-5, wherein the selective agent is cyanamide or an active derivative of cyanamide.

7. The method of any one of 1-6, wherein the selection marker is cyanamide hydratase.

8. The method of any one of 1-7, wherein the polynucleotide encoding the selection marker has been synthesized using plant preferred codons.

9. The method of 8, wherein the polynucleotide encoding the selection marker has been synthesized using maize preferred codons.

10. The method of any one of 1-9, wherein the DNA construct comprises in the following order: a first expression unit comprising the promoter active in the seed operably linked to the first recombination site operably linked to the polynucleotide encoding the selection marker; and a second expression unit comprising in the following order, a second promoter active in the plant operably linked to a polynucleotide of interest, and the second recombination site.

11. The method of any one of 1-9, wherein the DNA construct comprises in the following order: a first expression unit comprising the promoter active in the seed operably linked to the first recombination site operably linked to the polynucleotide encoding the selection marker; a second expression unit comprising in the following order, a second promoter active in the plant operably linked to a polynucleotide of interest; and a third expression unit comprising in the following order a third promoter active in the plant operably linked to the second recombination site.

12. The method of any of 1-11, wherein the DNA construct comprises the following operably linked components in the following order: the promoter active in the seed; an ATG start codon; the first recombination site; and the polynucleotide encoding the selection marker, wherein the polynucleotide encoding the selection marker lacks an operably linked promoter and an ATG start codon.

13. The method of any of 1-9, wherein the DNA construct comprises in the following order: a first expression unit comprising the following operably linked components, the promoter active in the seed, an ATG start codon, the first recombination site, and the polynucleotide encoding the selection marker, wherein the polynucleotide encoding the selection marker lacks an operably linked promoter and an ATG start codon; a second expression unit comprising in the following order, a second promoter active in the plant operably linked to a polynucleotide of interest; and, a third expression unit comprising in the following order, a third promoter active in the plant operably linked to the second recombination site.

14. The method of any one of 1-9, wherein the DNA construct comprises in the following order: a first expression unit comprising the following operably linked components, the promoter active in the seed, an ATG start codon, the first recombination site, and the polynucleotide encoding the selection marker, wherein the polynucleotide encoding the selection marker lacks an operably linked promoter and an ATG start codon; a second expression unit comprising in the following order, a second promoter active in the plant operably linked to a polynucleotide of interest; and, a third expression unit comprising, in the following order, a third promoter operably linked to a second ATG start codon operably linked to the second recombination site.

15. The method of any one of 1-14, wherein the plant further has stably incorporated into its genome a polynucleotide encoding a site specific recombinase.

16. The method of 15, wherein the site specific recombinase comprises a FLP recombinase, a Cre recombinase, a lambda integrase, a SSV1 integrase, or a phiC31 integrase.

17. The method of any one of 1-16, wherein at least one of the first or the second recombination sites are selected from the group consisting of a lox site, a mutant lox site, a FRT site, a mutant FRT site, an att site, and a mutant att site.

18. The method of 2, wherein the first and the second recombination site comprise a FRT site and a mutant FRT site.

19. The method of 2, wherein the first and the second recombination sites comprise mutant FRT sites.

20. The method of any one of 1-19, wherein the plant is a monocot.

21. The method of any one of 1-19, wherein the plant is a dicot.

22. The method of any one of 20 or 21, wherein the plant is maize, wheat, rice, sorghum, rye, oat, barley, millet, Brassica, alfalfa, sunflower, safflower, soybean, tobacco, cotton, or Arabidopsis.

23. A method of priming a seed comprising
(a) providing a seed having stably incorporated into its genome a DNA construct comprising, in the following order, a promoter active in the seed operably linked to a first recombination site operably linked to a polynucleotide encoding a selection marker that confers resistance to a selective agent; and
(b) contacting the seed with a priming matrix comprising an effective concentration of the selective agent for a time sufficient to produce a primed seed.

24. The method of 23, wherein the DNA construct comprises in the following order the promoter operably linked to the first recombination site operably linked the polynucleotide encoding the selection marker, and a second recombination site.

25. The method of 24, wherein the first and the second recombination sites are dissimilar and non-recombinogenic with each other.

26. The method of any one of 23-25, further comprising drying the primed seed.

27. The method of any one of 23-26, further comprising incubating the primed seed under germinating conditions.

28. The method of any one of 23-27, wherein the selective agent comprises an herbicide, a growth regulator, or an antibiotic.

29. The method of any one of 23-28, wherein the selective agent is selected from the group consisting of glufosinate, phosphinothricin, glyphosate, bromoxynil, methotrexate, imidazolinones, sulfonylureas, cyanamide, kanamycin, G418, neomycin, and hygromycin B.

30. The method of 29, wherein the selection marker is selected from the group consisting of Bar, phosphinothricin acetyltransferase (PAT), glyphosate oxidoreductase (GOX), 5-enolpyruvylshikimate-3-phosphate synthase (EPSPS), CP4, glyphosate N-acetyltransferase (GAT), bromoxynil nitrilase (BXN), dihydrofolate reductase, acetolactate synthase (ALS), cyanamide hydratase (CAH), neomycin phosphotransferase (nptil), aminoglycoside 3′-phosphotransferase (APH3′ II), and hygromycin phosphotransferase (hph).

31. The method of any one of 23-30, wherein the selective agent is cyanamide or an active derivative thereof.

32. The method of any one of 23-31, wherein the selection marker is cyanamide hydratase.

33. The method of any one of 24-32, wherein the DNA construct comprises in the following order: a first expression unit comprising the following operably linked components, the promoter active in the seed, the first recombination site, and the polynucleotide encoding the selection marker that confers resistance to the selective agent; and, a second expression unit comprising in the following order, a second promoter active in a plant operably linked to a polynucleotide of interest, and the second recombination site.

34. The method of any one of 24-32, wherein the DNA construct comprises in the following order: a first expression unit comprising the following operably linked components, the promoter active in the seed, the first recombination site, and the polynucleotide encoding the selection marker; a second expression unit comprising in the following order, a second promoter active in a plant operably linked to a polynucleotide of interest; and, a third expression unit comprising in the following order a third promoter active in the plant operably linked to the second recombination site.

35. The method of any one of 33-34, wherein the polynucleotide of interest encodes a second selection marker.

36. The method of 35, wherein the second selection marker confers resistance to a second selective agent.

37. The method of any one of 23-36, wherein the DNA construct comprises in the following order: a first expression unit comprising the following operably linked components, a promoter active in the seed, an ATG start codon, the first recombination site, and the polynucleotide encoding the selection marker, wherein the polynucleotide encoding the selection marker lacks an operably linked promoter and an ATG start codon.

38. The method of any one of 24-36, wherein the DNA construct comprises in the following order: a first expression unit comprising the following operably linked components, a promoter active in the seed, an ATG start codon, the first recombination site, and the polynucleotide encoding the selection marker, wherein the polynucleotide encoding the selection marker lacks an operably linked promoter and an ATG start codon; a second expression unit comprising, in the following order, a second promoter active in a plant operably linked to a polynucleotide of interest; and, a third expression unit comprising, in the following order, a third promoter active in the plant operably linked to the second recombination site.

39. The method of any one of 24-36, wherein the DNA construct comprises in the following order: a first expression unit comprising the following operably linked components, a promoter active in the seed, an ATG start codon, the first recombination site, and the polynucleotide encoding the selection marker, wherein the polynucleotide encoding the selection marker lacks an operably linked promoter and an ATG start codon; a second expression unit comprising in the following order, a second promoter active in a plant operably linked to a polynucleotide of interest; and, a third expression unit comprising in the following order, a third promoter active in the plant operably linked to a second ATG start codon operably linked to the second recombination site.

40. The method of any one of 23-36, wherein the seed further has stably incorporated into its genome a polynucleotide encoding a site specific recombinase.

41. The method of 40, wherein the site specific recombinase comprises a FLP recombinase, a Cre recombinase, a lambda integrase, a SSV1 integrase, or a phiC31 integrase.

42. The method of any one of 24-25, wherein at least one of the first or the second recombination sites are selected from the group consisting of a lox site, a mutant lox site, a FRT site, a mutant FRT site, an att site, and a mutant att site.

43. The method of any one of 24-25, wherein the first and the second recombination site comprise a FRT site and a mutant FRT site.

44. The method of any one of 24-45, wherein the first and the second recombination site comprise mutant FRT sites.

45. The method of any one of 23-44, wherein the seed is from a monocot.

46. The method of 45, wherein the monocot is maize, sorghum, barley, rice, oat, wheat, or millet.

47. The method of any one of 23-44, wherein the seed is from a dicot.

48. A method to identify a plant having a targeted insertion of a polynucleotide of interest in its genome comprising:
(a) providing a population of plants having stably incorporated into their genome a DNA construct comprising a promoter active in a seed operably linked to a target site, the target site comprising a first recombination site;
(b) introducing into the population of plants a transfer cassette comprising in the following order the first recombination site operably linked to a polynucleotide encoding a selection marker that confers resistance to a selective agent, wherein the polynucleotide is not operably linked to a promoter, and the polynucleotide of interest;
(c) providing a recombinase to the population of plants, wherein the recombinase recognizes and implements recombination at the first recombination site;
(d) contacting a population of seeds produced from the population of plants from step (c) with a priming matrix comprising an effective concentration of the selective agent for a time sufficient to produce a population of primed seeds; and,
(e) incubating the population of primed seeds under germination conditions, thereby identifying plants having the targeted insertion of the polynucleotide of interest.

49. A method to identify a plant having a targeted insertion of a polynucleotide of interest in its genome comprising:
(a) providing a population of plants having stably incorporated into their genome a DNA construct comprising a promoter active in a seed operably linked to a target site, the target site comprising a first and a second recombination site, wherein the first and the second recombination sites are dissimilar and non-recombinogenic with each other;
(b) introducing into the population of plants a transfer cassette comprising in the following order the first recombination site operably linked to a polynucleotide encoding a selection marker that confers resistance to a selective agent, wherein the polynucleotide is not operably linked to a promoter, the polynucleotide of interest, and the second recombination site;
(c) providing a recombinase to the population of plants, wherein the recombinase recognizes and implements recombination at the first and the second recombination site;
(d) contacting a population of seeds produced from the population of plants from step (c) with a priming matrix comprising an effective concentration of the selective agent for a time sufficient to produce a population of primed seeds; and,
(e) incubating the population of seeds under germination conditions, thereby identifying plants having the targeted insertion of the polynucleotide sequence of interest.

50. The method of any one of 48-49, wherein introducing in step (b) comprises sexual breeding.

51. The method of any one of 48-50, wherein the transfer cassette comprises in the following order: the first recombination site operably linked to the polynucleotide encoding the selection marker that confers resistance to the selective agent, wherein the polynucleotide is not operably linked to a promoter; a second promoter active in the plant operably linked to the polynucleotide of interest; and the second recombination site.

52. The method of any one of 48-51, wherein the transfer cassette comprises in the following order: the first recombination site operably linked to the polynucleotide encoding the selection marker that confers resistance to the selective agent, wherein the polynucleotide is not operably linked to a promoter; a second promoter active in the plant operably linked to the polynucleotide of interest; and a third promoter active in the plant operably linked to the second recombination site.

53. The method of any one of 48-52, wherein the DNA construct comprises the promoter active in the seed operably linked to an ATG start codon operably linked to the target site; and wherein the transfer cassette comprises in the following order, the first recombination site operably linked to a polynucleotide encoding a selection marker, wherein the polynucleotide lacks an operably linked promoter and an ATG start codon, and the polynucleotide of interest.

54. The method of any one of 48-53, wherein the DNA construct comprises the promoter active in the seed operably linked to an ATG start codon operably linked to the target site; and wherein the transfer cassette comprises in the following order, the first recombination site operably linked to a polynucleotide encoding a selection marker, wherein the polynucleotide lacks an operably linked promoter and an ATG start codon, and a second promoter active in the plant operably linked to the polynucleotide of interest.

55. A composition comprising a seed having stably incorporated into its genome a DNA construct comprising, in the following order, a promoter active in the seed operably linked to a first recombination site operably linked to a polynucleotide encoding a selection marker that confers resistance to a selective agent; and, a priming matrix comprising an effective concentration of the selective agent.

56. The composition of 55, wherein the DNA construct comprises in the following order, the promoter operably linked to the first recombination site operably linked to the polynucleotide encoding the selection marker, and a second recombination site.

57. The composition of 56, wherein the first and the second recombination sites are dissimilar and non-recombinogenic with each other.

58. The composition of any one of 55-57, wherein the DNA construct further comprises a polynucleotide of interest.

59. The composition of any one of 55-58, wherein the DNA construct further comprises a second selection marker.

60. The composition of 59, wherein the second selection marker confers resistance to a second selective agent.

61. A method to identify a maize plant having a DNA construct stably incorporated in its genome comprising
(a) providing a population of seeds, wherein at least one seed in the population has stably incorporated into its genome the DNA construct comprising, in the following order, a promoter active in the seed operably linked to a first recombination site operably linked to a polynucleotide encoding a selection marker that confers resistance to a selective agent, and a second recombination site;
(b) contacting the population of seeds with a priming matrix comprising an effective concentration of the selective agent for a time sufficient to produce a population of primed seeds, wherein the selective agent comprises cyanamide and the selection marker comprises cyanamide hydratase; and,
(c) incubating the population of primed seed under germination conditions, and thereby identifying plants having the DNA construct stably incorporated into their genome.

62. The method of any one of 1-55, or 61, wherein the priming matrix further comprises an additive selected from the group consisting of glycerol, Tween-20, and NP-40.

63. The composition of 60, wherein the priming matrix further comprises the second selective agent.

64. The composition of any one of 56-60 or 63, wherein the priming matrix further comprises an additive selected from the group consisting of glycerol, Tween-20, and NP-40.

65. A method to identify a plant having a DNA construct stably incorporated in its genome comprising:
(a) providing a population of seeds, wherein at least one seed in the population has stably incorporated into its genome the DNA construct comprising the following operably linked components in the following order:
   (i) a first expression unit comprising from 5′-3′: a first promoter active in the seed, a first recombination site, a first polynucleotide encoding a first selection marker that confers resistance to a first selective agent, and a first terminator active in a plant; and,
   (ii) a second expression unit comprising from 5′-3′: a second terminator active in a plant, a second polynucleotide encoding a second selection marker that confers resistance to a second selective agent, a second recombination site, and a second promoter active in the seed, wherein the first and the second selection markers confer resistance to different selective agents;
(b) contacting the population of seeds with a priming matrix comprising an effective concentration of the first and the second selection agents for a time sufficient to produce a population of primed seeds; and,
(c) incubating the population of primed seed under germination conditions, thereby identifying plants having the DNA construct stably incorporated into their genome.

66. The method of 65, wherein the first and the second recombination sites are dissimilar and non-recombinogenic with each other.

67. The method of any one of 65-66, wherein at least one of the first or the second selective agents comprises an herbicide, a growth regulator, or an antibiotic.

68. The method of any one of 65-67, wherein at least one of the first or the second selective agents is selected from the group consisting of glufosinate, phosphinothricin, glyphosate, bromoxynil, methotrexate, imidazolinones, sulfonylureas, cyanamide, kanamycin, G418, neomycin, and hygromycin B.

69. The method of any one of 65-67, wherein at least one of the first or the second selection markers is selected from the group consisting of Bar, phosphinothricin acetyltransferase (PAT), glyphosate oxidoreductase (GOX), 5-enolpyruvylshikimate-3-phosphate synthase (EPSPS), CP4, glyphosate N-acetyltransferase (GAT), bromoxynil nitrilase (BXN), dihydrofolate reductase, acetolactate synthase (ALS), cyanamide hydratase (CAH), neomycin phosphotransferase (nptII), aminoglycoside 3′-phosphotransferase (APH3′ II), and hygromycin phosphotransferase (hph).

70. The method of any one of 65-69, wherein either the first or the second selective agent is cyanamide or an active derivative of cyanamide.

71. The method of any one of 65-70, wherein either the first or the second selection marker is cyanamide hydratase.

72. The method of any one of 65-71, wherein at least one of the first or the second polynucleotide has been synthesized using plant preferred codons.

73. The method of 72, wherein at least one of the first or the second polynucleotide has been synthesized using maize preferred codons.

74. The composition of any one of 55-60, wherein the seed is from a plant selected from the group consisting of maize, wheat, rice, sorghum, rye, oat, barley, millet, Brassica, alfalfa, sunflower, safflower, soybean, tobacco, cotton, and Arabidopsis.

Compositions and methods to prime seeds are provided. For example, a method of priming a seed comprises providing a seed having stably incorporated into its genome a DNA construct comprising, in the 5′ to 3′ or 3′ to 5′ orientation, the following operably linked components: a promoter active in the seed, a first recombination site, and a polynucleotide encoding a selection marker that confers resistance to a selective agent. This seed is contacted with a priming matrix comprising an effective concentration of the selective agent for a time sufficient to produce a primed seed. Seeds having such a DNA construct can be generated in a variety of ways. In some examples, the seed having such a DNA construct is a targeted seed. In some methods the DNA construct further comprises at least a second recombination site. In some examples the DNA construct further comprises a polynucleotide of interest. In some examples the DNA construct further comprises a second selection marker. In some examples the first and the second recombination sites are dissimilar and non-recombinogenic with each other. In some examples the seed is dried after priming. In some examples the method further comprises incubating the primed seed under germination conditions. In some examples the seed priming methods and compositions provide efficient isolation and/or identification of seeds/plants having such DNA constructs. In some examples, the priming methods allow for the identification of a seed having an activated selectable marker.

Using traditional methods, seed expressing the selection marker would be segregated based on leaf painting and/or spraying, followed by molecular analysis such as PCR, sequencing, ELISA, or any combination of these or similar techniques. The methods herein expose the seed to the appropriate selective agent during the priming stage, which inhibits germination in the absence of adequate resistance, providing easy, fast, and efficient screening. For example it allows a large population seeds to be screened without segregating, leaf painting, spraying, and/or molecular analyses. Further characterization and analyses can be done, including but not limited to PCR, sequencing, Southern blots, Northern blots, Western blots, ELISA, mapping, agronomic evaluations, and the like.

Compositions include a mixture comprising: a seed having stably incorporated into its genome a DNA construct comprising the following operably linked components, a promoter active in the seed, a first recombination site, and a polynucleotide encoding a selection marker that confers resistance to a selective agent; and, a priming matrix comprising an effective concentration of the selective agent. In some examples the DNA construct further comprises at least a second recombination site. In some examples the first and the second recombination sites are dissimilar and non-recombinogenic with each other. In some examples the DNA construct further comprises at least one polynucleotide of interest. In some examples the DNA construct further comprises a second selection marker. In some examples the priming matrix further comprises a second selection agent, a surfactant, a fungicide, a preservative, and/or another compound.

The methods provide for the identification of plants having stably incorporated into their genome a DNA construct comprising in the 5′ to 3′ or 3′ to 5′ orientation the following operably linked components: a promoter active in the seed, a first recombination site, and a polynucleotide encoding a selection marker that confers resistance to a selective agent. In some examples the method comprises providing a population of plants having stably incorporated into their genome a DNA construct comprising a promoter active in the seed operably linked to a target site, the target site comprising a first recombination site; providing to the population of plants a transfer cassette comprising the first recombination site operably linked to a polynucleotide encoding a selection marker that confers resistance to a selective agent, wherein the polynucleotide is not operably linked to a promoter; and, providing to the population of plants a recombinase wherein the recombinase recognizes and implements recombination between the first recombination sites. A population of seed can be generated from this population of plants, this seed can be contacted with a priming matrix comprising an effective concentration of the selective agent for a time sufficient to produce a population of primed seed. In some examples the primed seed are then dried. In some examples the primed seed are further incubated under germination conditions to identify plants having the DNA construct. In some examples, DNA construct further comprises at least a second recombination site. In some examples, the first and the second recombination sites are dissimilar and non- recombinogenic with each other. In some examples the DNA construct further comprises a polynucleotide of interest. In some examples the polynucleotide of interest encodes a second selection marker. In some examples the second selection marker confers resistance to a second selective agent. Seeds having such a DNA construct can be generated in a variety of ways.

In some instances, the seed having such a DNA construct is a targeted seed, wherein a targeted seed or plant comprises a DNA construct that was generated and/or manipulated via a site-specific recombination system. In one example, the generation of the targeted seed comprises: providing a population of plants having stably incorporated into their genome a DNA construct comprising a promoter active in the seed operably linked to a target site, the target site comprising a first recombination site; providing to the population of plants a transfer cassette comprising the first recombination site operably linked to a polynucleotide encoding a selection marker that confers resistance to a selective agent, wherein the polynucleotide is not operably linked to a promoter; and, providing to the population of plants a recombinase wherein the recombinase recognizes and implements recombination between the first recombination sites. In some examples a population of seed from the population of plants is contacted with a priming matrix comprising an effective concentration of the selective agent for a time sufficient to produce a population of primed seeds. In some examples the primed seed is then dried. In some examples the primed seed is further incubated under germination conditions and plants having the targeted insertion of the DNA construct identified. The population of plants obtained comprises targeted plants having the transfer cassette integrated at the target site, and plants in which the transfer cassette integrated randomly in the genome. The methods and compositions provide efficient identification and/or isolation of the targeted plants. In one example, the transfer cassette can be provided to the plant having the target site by a sexual cross. In some examples the target site further comprises a second recombination site, wherein the first and the second recombination sites are dissimilar and non-recombinogenic with each other. In some examples the transfer cassette further comprises the second recombination site, wherein the first and second recombination sites are dissimilar and non-recombinogenic with each other, and correspond to the recombination sites in the target site, wherein a recombinase is provided which recognizes and implements recombination at the first and second recombination sites. In some examples the target site comprises a first recombination site, and the transfer cassette comprises a second recombination site, wherein the first and the second recombination sites are dissimilar and recombinogenic with each other. In some examples the recombinase provided comprises a biologically active variant and/or fragment of the recombinase. In some examples, the recombination product of the dissimilar and recombinogenic first and second recombination sites comprises a modified first and modified second recombination sites which are dissimilar and non-recombinogenic with each other, thereby inhibiting the reverse recombination reaction. The dissimilar and recombinogenic sites can have at least one nucleotide change in any region of the site, including but not limited to a recombinase binding element, spacer region, or combination thereof. For example, mutant lox sites with introduced nucleotide changes into the left 13 bp element (LE mutant lox site) or the right 13 bp element (RE mutant lox site) have been used (Albert et al. (1995) Plant J 7:649-659). Recombination between the LE mutant lox site and the RE mutant lox site produces the wild-type loxP site and a LE/RE mutant site that is poorly recognized by Cre, resulting in a stable integration event. See also, for example, Araki et al. (1997) Nucleic Acids Res 25:868-872. In some examples, a recombination system is used wherein the reverse reaction is inhibited, for example, an SSV1, lambda, or phiC31 integrase system using Int recombinase and appropriate att sites.

In some examples, at least one polynucleotide of interest can be included in a construct, for example the target site, transfer cassette, and/or DNA construct. Any polynucleotide of interest can be used, including markers, a recombinase, trait genes, regulatory sequences, insulators, operators, repressors, replication origins, binding regions, recognition sites, templates, activators, silencing constructs, and the like. A change in phenotype may be provided by expression of heterologous products, increased expression of endogenous products in plants, and/or reduced expression of one or more products, or any combination thereof.

Various methods can be used to generate a targeted seed or plant. Once the targeted seed is generated, priming with the appropriate selective agent is used to identify targeted seed having the desired DNA construct. Methods are also provided to identify a plant having a targeted insertion of a polynucleotide of interest in its genome. In some examples the method comprises providing a population of plants having stably incorporated into their genome a DNA construct comprising in the 5′ to 3′ or 3′ to 5′ orientation a promoter active in the seed operably linked to a target site, wherein the target site comprises a first and a second recombination site, wherein the first and the second recombination sites are dissimilar and non-recombinogenic with each other; introducing into the population of plants a transfer cassette comprising in the 5′ to 3′ or 3′ to 5′ orientation the first recombination site, a polynucleotide encoding a selection marker that confers resistance to a selective agent, and the second recombination site, wherein the polynucleotide encoding the selection marker is not operably linked to a promoter; providing to the population of plants a recombinase, wherein the recombinase recognizes and implements recombination at the first and the second recombination sites; contacting a population of seeds produced from the population of plants described above with a priming matrix comprising an effective concentration of the selective agent for a time sufficient to produce a population of primed seed; and, incubating the population of primed seed under germination conditions, thereby identifying plants having the targeted insertion of the polynucleotide. In some examples the first and the second recombination sites are dissimilar and recombinogenic with each other. In some examples the recombinase provided comprises a biologically active variant and/or fragment of the recombinase.

The plants, cells, and/or seeds employed can have stably incorporated into their genome a DNA construct comprising in the 5′ to 3′ or 3′ to 5′ orientation a promoter active in the seed operably linked to a first recombination site operably linked to a polynucleotide encoding a selection marker that confers resistance to a selective agent. In some examples the DNA construct further comprises at least a second recombination site. In some examples, the first and the second recombination sites are dissimilar and non-recombinogenic with each other. In some examples the first and the second recombination sites are dissimilar and recombinogenic with each other. In some examples the DNA construct further comprises a polynucleotide of interest.

Several examples of target sites, transfer cassettes, and DNA constructs include, but are not limited to those shown in TABLE 1. The letters and/or numbers are for identification only, and not meant to imply any association between the various constructs.

TABLE 1
Target sites

| | |
|---|---|
| A | P1::RSa |
| B | P1::RSa::NT1 |
| C | P1::RSa::NT1::T1 |
| D | P1::ATG::RSa |
| E | P1::ATG::RSa::NT1(no ATG) |
| F | P1::ATG::RSa::NT1(no ATG)::T1 |
| G | P1::RSa::P2 |
| H | P1::ATG::RSa::P2 |
| I | P1::RSa::ATG::P2 |
| J | P1::ATG::RSa::ATG::P2 |
| K | P1::RSa::spacer::RSb |
| L | P1::ATG::RSa::spacer::RSb |
| M | P1::RSa::NT1::T1-RSb |
| N | P1::RSa::NT1::T1-RSb::P2 |
| O | P1::ATG::RSa::NT1(no ATG)::T1-RSb::P2 |
| P | P1::RSa::NT1::T1-RSb::P2::NT2::T2 |
| Q | P1::ATG::RSa::NT1(no ATG)::T1-RSb::P2::NT2::T2 |
| R | P1::RSa::NT1::T1-P2::NT2::T2-RSb |
| S | P1::RSa::NT1::T1-P2::NT2::T2-P3::RSb |
| T | P1::ATG::RSa::NT1(no ATG)::T1-P2::NT1::T2::RSb |
| U | P1::ATG::RSa::NT1(no ATG)::T1-P2::NT1::T3-P3::ATG::RSb |
| V | P1::RSa::NT1::T1-P2::NT2::T2-P3::RSb::S2::T3-P4::NT3::T4::RSc |
| W | P1::ATG::RSa::NT1(no ATG)::T1-P2::NT2::T2-P3::RSc |

Transfer Cassettes

| | |
|---|---|
| 1 | RSa::S1 |
| 2 | RSa::S1::T1 |
| 3 | RSa::S1(no ATG) |
| 4 | RSa::S1(no ATG)::T1 |
| 5 | T2::S2::RSa::S1::T1 (circular) |
| 6 | T2::S2::RSa::S1(no ATG)::T1 (circular) |
| 7 | T2::S2(no ATG)::RSa::S1::T1 (circular) |
| 8 | T2::S2(no ATG)::RSa::S1(no ATG)::T1 |
| 9 | RSa::S1::T1-P2::NT1 |
| 10 | RSa::S1::T1-P2::NT1::T2 |
| 11 | RSa::S1(no ATG)::T1-P2::NT1 |
| 12 | T3::S2::RSa::S1::T1-P2::NT1::T2 (circular) |
| 13 | RSa::S1-RSb |
| 14 | RSa::S1::T1-RSb |
| 15 | RSa::S1::T1-P2::NT1::T2-RSb |
| 16 | RSa::S1::T1-P2::NT1::T2-P3::RSb |
| 17 | RSa::S1(no ATG)::T1-P2::NT1::T2::RSb |
| 18 | RSa::S1::T1-P2::NT1::T3-P3::ATG::RSb |
| 19 | RSa::S1(no ATG)::T1-P2::NT1::T3-P3::ATG::RSb |
| 20 | RSa::S1::T1-P2::NT1::T2-P3::RSb::S2::T3-P4::NT2::T4::RSc |
| 21 | RSa::S1::T1-P2::NT1::T2-P3::RSc::S2::T3-P4::NT2::T4::RSb |
| 22 | RSa::S1(no ATG)::T1-P2::NT1::T2-P3::ATG::RSb::S2(no ATG)::T3-P4::NT2::T4::RSc |

DNA constructs

| | |
|---|---|
| a | P1::RSa::S1 |
| b | P1::RSa::S1::T1 |
| c | P1::ATG::RSa::S1(no ATG) |
| d | P1::ATG::RSa::S1(no ATG)::T1 |
| e | P1::RSa::S1::T1-T2::S2::RSa::P2 |
| f | P1::ATG::RSa::S1(no ATG)::T1-T2::S2::RSa::P2 |
| g | P1::RSa::S1::T1-T2::S2(no ATG)::RSa::ATG::P2 |
| h | P1::ATG::RSa::S1(no ATG)::T1-T2::S2(no ATG)::RSa::ATG::P2 |
| i | P1::RSa::S1::T1-P2::NT1 |
| j | P1::RSa::S1::T1-P2::NT1::T2 |
| k | P1::ATG::RSa::S1(no ATG)::T1-P2::NT1 |
| l | P1::RSa::S1::T1-P2::NT1::T2-T3::S2::RSa::P3 |
| m | P1::RSa::S1-RSb |
| n | P1::RSa::S1::T1-RSb |
| o | P1::RSa::S1::T1-P2::NT1::T2-RSb |
| p | P1::RSa::S1::T1-P2::NT1::T2-P3::RSb |
| q | P1::ATG::RSa::S1(no ATG)::T1-P2::NT1::T2::RSb |
| r | P1::RSa::S1::T1-P2::NT1::T3-P3::ATG::RSb |
| s | P1::ATG::RSa::S1(no ATG)::T1-P2::NT1::T3-P3::ATG::RSb |
| t | P1::RSa::S1::T1-P2::NT1::T2-P3::RSb::S2::T3-P4::NT2::T4::RSc |
| u | P1::ATG::RSa::S1(no ATG)::T1-P2::NT1::T2-P3::ATG::RSb::S2(no ATG)::T3- |
| | P4::NT2::T4::RSc |

TABLE 1:
P = promoter;
RS = recombination site;
S = selection marker;
T = terminator region;
NT = polynucleotide of interest;
ATG = start codon;
no ATG indicates that the coding region lacks the ATG start codon;
the symbol :: indicates an operable linkage or fusion between adjacent elements; and
ATG::RS indicates an operable in-frame fusion to produce a properly expressed functional gene product.

In some examples the plant comprises a DNA construct comprising a first expression unit comprising the following operably linked components in 5′ to 3′ or 3′ to 5′ orientation: a promoter active in a seed, a first recombination site, and a selection marker. In some examples, a termination region is operably linked to the selection marker. In some examples the DNA construct further comprises a second promoter. In some examples, a polynucleotide of interest is operably linked to the second promoter, optionally with a termination region. In some examples, the DNA construct can further comprise at least a second recombination site. In some examples, the first and the second recombination sites are identical. In further examples, the first and the second recombination sites are dissimilar and non-recombinogenic with each other.

In some examples, the plant comprises a DNA construct comprising a first expression unit comprising the following operably linked components in 5′ to 3′ or 3′ to 5′ orientation: promoter active in a seed, an ATG codon, a first recombination site, and a selection marker, wherein the selection marker lacks an ATG start codon (P1::ATG::RSa::S1 (no ATG)). In some examples, a termination region is operably linked to the selection marker. In some examples the DNA construct further comprises a second promoter. In some examples, a polynucleotide of interest is operably linked to the second promoter. Optionally the polynucleotide of interest is operably linked to a termination region. In some examples, the DNA construct further comprises at least a second recombination site. In some examples, the first and the second recombination sites are identical. In further examples, the first and the second recombination sites are dissimilar and non-recombinogenic with each other.

In some examples, the plant comprises a DNA construct comprising a first and a second expression unit, wherein the first expression unit comprises following operably linked components in 5′ to 3′ or 3′ to 5′ orientation: a promoter active in a seed, a first recombination site, and a first selection marker, optionally linked to a termination region, and the second expression cassette comprises the following operably linked components in 5′ to 3′ or 3′ to 5′ orientation: a termination region, a second selection marker, a second recombination site, and a second promoter. In some examples, the first and the second recombination sites are identical. In further examples, the first and the second recombination sites are dissimilar and non-recombinogenic with each other. In some examples, at least one promoter is operably linked to an ATG, wherein the operably linked selection marker lacks an ATG start codon. In some examples the DNA construct further comprises a promoter operably linked to a polynucleotide of interest.

In some instances, the plant comprises a DNA construct comprising a first and a second expression unit, where the first expression unit comprises a first promoter active in a seed operably linked to a first recombination site operably linked to a selection marker. The second expression unit comprises a second promoter active in the plant operably linked to a polynucleotide of interest operably linked to a second recombination site. In some examples, the first and the second recombination sites are dissimilar and non-recombinogenic with each other. Optionally, at least one terminator region may be operably linked to the first and/or second expression unit.

In other instances, the DNA construct comprises a first, a second, and a third expression unit. The first expression unit comprises the following operably linked components in 5′ to 3′ or 3′ to 5′ orientation: a promoter active in a seed, a first recombination site, and a selection marker. The second expression unit comprises a second promoter active in the plant operably linked to a polynucleotide of interest. The third expression cassette comprises a third promoter active in the plant operably linked to a second recombination site. In some examples, the first and the second recombination sites are dissimilar and non-recombinogenic with each other. Optionally, at least one terminator region may be operably linked to the first, second, and/or third expression unit.

In other examples, the DNA construct comprises a first and a second expression unit. The first expression unit comprises the following operably linked components in 5′ to 3′ or 3′ to 5′ orientation: a promoter active in the seed, an ATG start codon, a first recombination site, and a selection marker, wherein the selection marker lacks an ATG start codon. The second expression unit comprises a second promoter active in the plant operably linked to a polynucleotide of interest, and a second recombination site. In some cases, the first and the second recombination sites are dissimilar and non-recombinogenic with each other. Optionally, at least one terminator region may be operably linked to the first and/or second expression unit.

In other examples, the DNA construct comprises a first, a second, a third, and a fourth expression unit. The first expression unit comprises the following operably linked components in 5′ to 3′ or 3′ to 5′ orientation: a promoter active in the seed, an ATG start codon, a first recombination site, and a selection marker, wherein the selection marker lacks an ATG start codon. The second expression unit comprises a second promoter active in a plant operably linked to a polynucleotide of interest. The third expression unit comprises the following operably linked components in 5′ to 3′ or 3′ to 5′ orientation: a third promoter active in a plant, an ATG start codon, a second recombination site, and a second selection marker, wherein the second selection marker lacks an ATG start codon. The fourth expression unit comprises the following operably linked components in 5′ to 3′ or 3′ to 5′ orientation: a fourth promoter active in the plant, a second polynucleotide of interest, and a third recombination site. In some examples, the first, the second, and/or the third recombination sites are dissimilar and non-recombinogenic with each other. Optionally, at least one terminator region may be operably linked to the first, second, third and/or fourth expression unit.

In other examples, an expression unit can comprise a promoter operably linked to a first polynucleotide and a second polynucleotide such that the single promoter drives expression of both polynucleotides. Optionally, at least one terminator region may be operably linked to the first, and/or second polynucleotide sequence.

In some examples, target sites and transfer cassettes are used to manipulate, exchange, excise, invert, alter, and/or introduce the DNA construct. In some examples, the recombination sites are dissimilar and non-recombinogenic with each other. In some examples, the recombination sites are dissimilar and recombinogenic with each other. One or more intervening sequences may be present between the recombination sites of the target site and/or transfer cassette. In some examples, the recombination sites of the target site and/or transfer cassette may be directly contiguous with the selection marker and/or a polynucleotide of interest. Intervening sequences of interest include linkers, adapters, selectable markers, additional polynucleotides of interest, promoters, regulatory sequences, insulators, binding regions, recognition sites, repressors, operators, activators and/or other sites/sequences that aid in vector construction, expression, or analysis. In addition, the recombination site(s) of the target site can be located and/or oriented in various positions, including, for example, within intronic sequences, coding sequences, or untranslated regions. In certain examples, the recombination sites can be contained within the selectable marker and/or the polynucleotide of interest, for example within an intron, coding sequence, or untranslated region. In some examples, the plant may comprise multiple target sites at one or more locations in the genome. When more than one target site is at one locus in the genome, multiple manipulations of this target locus in the plant are available. In some examples, the genome of the plant having the target site may also comprise an expression cassette comprising a polynucleotide encoding an appropriate recombinase. In another example, the target site itself comprises a nucleotide sequence encoding a recombinase. Once a target site has been established within a genome it is possible to subsequently add, remove, and/or alter sites through recombination, for example, additional recombination sites may be introduced by incorporating such sites within the nucleotide sequence of the transfer cassette (see, e.g. WO99/25821).

Multiple genes or polynucleotides of interest can be stacked, ordered, and/or manipulated at a precise genomic location in the plant. For example, the DNA construct can comprise the following components: P1::RSa::S1::T1-P2::NT1::T2-P3::RSb-RSc. In certain examples, P3::RSb allows priming/selection to be repeated by introducing a different second selection marker. For example, once the construct is incorporated into the genome, a transfer cassette comprising the following components could be introduced: RSb::S2::T3-P4::NT2::T4-RSc. The priming methods can then be used to select seed expressing the second selection marker, or the first and the second marker. In this manner, multiple sequences can be stacked at predetermined locations. Various alterations can be made to stack the polynucleotides of interest in the genome of the plant. The targeted seeds produced can have a simple integration pattern comprising integration exclusively or predominantly at the target site with no or very few random insertions elsewhere in the genome. Methods for determining the integration patterns are known and include, for example, Southern blot analysis and RFLP analysis.

The activity of various promoters at a characterized location in the plant genome can be determined, and compared. Further, the activity, expression pattern, and/or expression level of a polynucleotide of interest can be determined. For example, a method for assessing promoter activity in a plant comprises introducing into the plant a transfer cassette comprising a first recombination site operably linked to a selection marker, wherein the selection marker is not operably linked to a promoter, a second promoter active in the plant operably linked to a polynucleotide of interest and the second recombination site. The plant further comprises in its genome a DNA construct comprising a first promoter active in the seed operably linked to a target site comprising the first and the second recombination site, wherein the first and the second recombination sites are dissimilar and non-recombinogenic with each other. A recombinase is provided, wherein the recombinase recognizes and implements recombination at the first and the second recombination sites. Seeds are then primed with an appropriate selective agent to identify targeted seeds wherein the transfer cassette has integrated at the target site. Such targeted seeds, or plants derived therefrom, can further be tested to assess the promoter activity of the second promoter. Once the activity of the promoter is characterized, additional transfer cassettes can be employed to allow the characterized promoter to drive expression of the polynucleotide of interest. In some instances, the activity of the second promoter can further be compared to the activity of the first promoter. Plant lines having such characterized promoters can be engineered so that polynucleotides of interest can be operably linked to the promoter, and thereby expressed in a desired manner. In some examples the recombinase provided comprises a biologically active variant and/or fragment of the recombinase.

The DNA constructs, transfer cassettes, and/or target sites can be designed to minimize or eliminate expression resulting from random integration of DNA sequences into the genome of a plant, for example, the transfer cassettes can be designed without either of a promoter or an ATG start codon operably linked to the selection marker. The target site comprises a promoter operably linked to a recombination site which is further operably linked to an ATG translation start site. Random integration of the transfer cassette is unlikely to produce expression of the selection marker, since the transfer cassette would need to randomly integrate behind an endogenous promoter region, an ATG start site, and in the correct reading frame.

A plant is provided having stably incorporated into its genome a DNA construct comprising the following operably linked components in the following order: a first promoter active in the seed, an ATG start codon, and a target site comprising a first recombination site. A transfer cassette is introduced into the plant comprising, in the following order: the first recombination site operably linked to a polynucleotide encoding a selection marker, wherein the polynucleotide encoding the selectable marker lacks an ATG start codon, and the polynucleotide encoding the selectable marker not operably linked to a promoter. A recombinase is provided that recognizes and implements recombination between the first recombination sites, which results in the selection marker being operably linked to the first promoter and the ATG start codon of the target site. Expression of the selection marker is controlled by the first promoter, therefore priming with the appropriate selective agent can be used to identify the targeted seed. In some examples the target site and/or the transfer cassette further comprises at least a second recombination site. In some examples the first and the second recombination sites are dissimilar and non-recombinogenic with each other. In some examples the first and the second recombination sites are dissimilar and recombinogenic with each other. In some examples the transfer cassette further comprises a second promoter operably linked to a polynucleotide of interest. In some examples, plants comprising the target site are crossed with plants comprising the transfer cassette to generate seeds/plants comprising the DNA construct. In some examples the recombinase comprises a biologically active variant and/or fragment of the recombinase.

Seed dormancy is a unique form of developmental arrest utilized by most plants to temporally delay germination and optimize progeny survival. During seed dormancy, moisture content and respiration rate are dramatically lowered. The initial step to break seed dormancy is the uptake (imbibition) of water to increase respiration and mobilization of starch reserves required for germination. The water uptake is triphasic, including an initial rapid period (phase I), followed by a pre-germination plateau (phase II) in which water uptake is slower and less intense than in phase I. In phase III, there is a subsequent increase in water uptake which results in the growth of the embryonic axis, radicle emergence, and resumption of growth, i.e., germination. The three phases are separated temporally, and a seed that has entered and/or completed phase I and II is said to be a primed seed, that is, primed for phase III: germination. Seed priming enables faster and more uniform germination upon sowing or planting of seed. Priming provides the option of simultaneously treating the seed with fungicide, preservatives, or other agents, to provide protection during processing, after sowing, and/or to prolong viable storage time. Seed priming allows the seeds to begin the pre-germinative metabolic processes, and then arrests the seeds at the pre-germinative stage. The amount of water absorbed is carefully controlled to avoid germination or seed ageing. Once the seed has absorbed sufficient water, the seed can be held at that water content, or dried back to the original water content for storage. Primed seeds can be sown directly without drying whereupon they germinate faster than seeds which have been primed and dried.

A priming matrix is a composition comprising an effective concentration of a selective agent and having an effective osmotic potential. An effective concentration of a selective agent is sufficient to prevent or significantly reduce the germination of seed that do not express the corresponding selection marker. An effective concentration of a selective agent comprises a concentration that prevents the germination of at least 70%, 75%, 80%, 85%, 90%, 91%, 92%, 93%, 94%, 95%, 96%, 97%, 98%, 99%, or 100% of a population of seeds that do not express the corresponding selection marker. An effective osmotic potential is an osmotic potential that lowers the water potential allowing or causing water to move into the seed to a level sufficient to prime the seed. Seeds germinate when water potential reaches a critical physiological level which varies between plant species, but typically falls between 0 and −2 Mpa. Many priming matrices that provide an appropriate osmotic potential are available, including water, water with at least one solute, solid matrices, and the like. For example, the priming matrix may comprise an aerated solution of osmotic material, such as polyethylene glycol (PEG) (see U.S. Pat. No. 5,119,598), glycerol, mannitol, and the like. Alternatively, seeds may be primed using a solid matrix. A solid matrix material should have a high water holding capacity to allow seeds to imbibe. In this method, the priming matrix can comprise an absorbent medium such as clay, vermiculite, perlite, saw dust, corn cobs, and/or peat to absorb water and then transfer it to the seed (see U.S. Pat. No. 4,912,874). The extent of hydration is controlled by altering the water content of the medium and the medium/seed ratio. Methods are also known to imbibe seeds in a slurry of PEG 6000 and vermiculite, or other matrices (Peterson (1976) Sci Hort 5:207-214; and U.S. Pat. No. 5,628,144). In still other methods, priming employs a semi-permeable membrane that mediates the transfer of water from a solution of set osmotic pressure to the seed (see, U.S. Pat. No. 5,873,197). In other methods, ultrasonic energy can be used to assist in the priming process (see, e.g., U.S. Pat. No. 6,453,609). Optionally a variety of additives, chemicals, and/or compounds can be included in the priming matrix, including surfactants, additional selective agents, fungicides, agents to modify osmotic potential, osmotic protectants, agents to aid drying or protect the seed during drying, agents to enhance seed processing, agents to extend storage shelf life, agents to enhance coating and/or perfusion, agents to enhance germination of the seed, and the like. Fungicides can be included in the priming matrix, for example, thiram, captan, metalaxyl, pentachloronitrobenzene, fenaminosulf, bactericides or other preservatives. In addition, various growth regulators/hormones, such as gibberellins/gibberellic acid, cytokinins, inhibitors of abscissic acid, 2-(3,4-dichlorophenoxy)triethylamine (DCPTA), potassium nitrate, and ethephon can also be present in the priming matrix. Other optional agents include glycerol, polyethylene glycol, mannitol, DMSO, Triton X-100, Tween-20, NP-40, ionic compounds, non-ionic compounds, surfactants, detergents, and the like. A time sufficient to produce a primed seed allows pre-germinative metabolic processes to take place within the seed up to any level including that immediately preceding radicle-emergence. The time to produce a primed seed is dependent on the specific seed variety, its state or condition, and the water potential of the priming matrix. While typical water amounts and media water potentials for given seed types are already generally known for some seeds, it is frequently best to test a small sample of a new seed over a readily determined range of osmotic potentials and temperatures to determine what conditions of temperature, water potential, and time provide appropriate imbibing of the seed and resultant pre-germination events. The temperature at which the priming methods are carried out may vary with the seeds to be treated, but typically is between 18° C. to 30° C. The primed seeds may be retained in the priming matrix through germination as denoted by radical emergence. Seed produced by this method may be further dried as in U.S. Pat. No. 4,905,411.

Methods to determine if a seed has been primed are known. For example, optimization of priming treatments can be performed by carrying out germination assays. See, for example, Jeller et al. (2003) Braz J Biol 63:61-68. In addition, molecular markers of germination and/or priming are known. See, for example, Job et al. (2000) Seed Biology: Advances and Applications, Eds. Black et al., CABI International, Wallingford, UK, pp. 449-459; De Castro et al. (2000) Plant Physiol 122:327-335; Bradford et al. (2000) Seed Biology: Advances and Applications, Eds. Black et al., CABI International, Wallingford, UK pp. 221-251; and Gallardo et al. (2001) Plant Physiol 126:835-848.

After priming, the seeds may be allowed to germinate, or the primed seeds can be dried. The appropriate conditions (temperature, relative humidity, and time) for the drying process will vary depending on the seed and can be determined empirically. See, for example, Jeller et al. (2003) Braz J Biol 63:61-68. Drying primed seed includes a superficial drying of the seed or, alternatively, drying the seed back to its original water content. The dried seeds can be immediately germinated or can be stored under appropriate conditions. Germination conditions for various seed are known. One factor in determining appropriate germination conditions is the threshold germination temperature range, which is the range of temperatures for a species within which seeds of that species will germinate at a predetermined moisture level and with adequate oxygen. Another factor is the threshold germination moisture range, which is the range of moisture for a species within which seeds of the species will germinate at a given temperature and with adequate oxygen. Threshold germination temperature range and/or threshold germination moisture range values are known for various seeds, as are methods to empirically determine these conditions for any given seed and variety.

In some examples, the population of primed seeds is incubated under germination conditions. Primed seed expressing the selection maker are resistant to the selective agent and will germinate to produce a viable plant. Primed seeds not expressing the selection marker will not be resistant to the selection agent and will not germinate, or germinate at a significantly lower frequency, or will germinate only to produce a coleoptile and then die, or will germinate with an abnormal phenotype and typically die prior to the V2 stage of development. Plants expressing the selection marker will thereby be identified and/or selected.

The methods and compositions further employ elements from recombination systems, such as recombinases and recombination sites, for example in a DNA construct, a target site, and/or a transfer cassette. A target site comprises a polynucleotide integrated into the genome comprising a promoter operably linked to at least one recombination site. A transfer cassette comprises at least a first recombination site operably linked to a polynucleotide encoding a selection marker, and optionally a polynucleotide of interest, wherein the first recombination site is recombinogenic with a recombination site in the target site. A targeted seed or plant has stably incorporated into its genome a DNA construct that has been generated and/or manipulated through the use of a recombination system. Site-specific recombination methods that result in various integration, alteration, exchange, replacement, and/or excision events to generate the recited DNA construct can be employed to generate a targeted seed. See, e.g., WO99/25821, WO99/25854, WO99/25840, WO99/25855, WO99/25853, WO99/23202, WO99/55851, WO01/07572, WO02/08409, and WO03/08045.

A recombinase is a polypeptide that catalyzes site-specific recombination between its compatible recombination sites, and includes naturally occurring recombinase sequences, variants, and/or fragments that retain activity. A recombination site is a nucleotide sequence that is specifically recognized by a recombinase enzyme, and encompasses naturally occurring recombination site sequences, variants, and/or fragments that retain activity. For reviews of site-specific recombinases, see Sauer (1994) Curr Op Biotech 5:521-527; Sadowski (1993) FASEB 7:760-767; Groth & Calos (2004) J Mol Biol 335:667-678; and Smith & Thorpe (2002) Mol Microbiol 44:299-307. Any recombination system, or combination of systems, can be used including but not limited to recombinases and recombination sites from the Integrase and Resolvase families, biologically active variants and fragments thereof, and/or any other naturally occurring or recombinantly produced enzyme or variant thereof that catalyzes conservative site-specific recombination between specified recombination sites, and naturally occurring or modified recombination sites or variants thereof that are specifically recognized by a recombinase to generate a recombination event.

The recombination sites employed can be corresponding sites or dissimilar sites. Corresponding recombination sites, or a set of corresponding recombination sites, are sites having an identical nucleotide sequence. A set of corresponding recombination sites, in the presence of the appropriate recombinase, will efficiently recombine with one another. Dissimilar recombination sites have a distinct sequence, comprising at least one nucleotide difference as compared to each other. The recombination sites within a set of dissimilar recombination sites can be either recombinogenic or non-recombinogenic with respect to one other. Each recombination site within the set of dissimilar sites is biologically active and can recombine with an identical site. Recombinogenic sites are capable of recombining with one another in the presence of the appropriate recombinase. Recombinogenic sites include those sites where the relative excision efficiency of recombination between the recombinogenic sites is above the detectable limit under standard conditions in an excision assay as compared to the wild type control, typically, greater than 2%, 5%, 10%, 20%, 50%, 100%, or greater. Non-recombinogenic sites will not recombine with one another in the presence of the appropriate recombinase, or recombination between the sites is not detectable. Non-recombinogenic recombination sites include those sites that recombine with one another at a frequency lower than the detectable limit under standard conditions in an excision assay as compared to the wild type control, typically, lower than 2%, 1.5%, 1%, 0.75%, 0.5%, 0.25%, 0.1%, 0.075, 0.005%, or 0.001%. Any suitable non-recombinogenic recombination sites may be utilized, including a FRT site or active variant thereof, a lox site or active variant thereof, an att site or active variant thereof, any combination thereof, or any other combination of non-recombinogenic recombination sites. Directly repeated recombination sites in a set of recombinogenic recombination sites are arranged in the same orientation, recombination between these sites results in excision of the intervening DNA sequence. Inverted recombination sites in a set of recombinogenic recombination sites are arranged in the opposite orientation, recombination between these sites results in inversion of the intervening DNA sequence.

The Integrase family of recombinases has over one hundred members and includes, for example, FLP, Cre, Int, and R. For other members of the Integrase family, see for example, Esposito et al. (1997) Nucleic Acids Res 25:3605-3614; Nunes-Duby et al. (1998) Nucleic Acids Res 26:391-406; Abremski et al. (1992) Protein Eng 5:87-91; Groth & Calos (2004) J Mol Biol 335:667-678; and Smith & Thorpe (2002) Mol Microbiol 44:299-307. Other recombination systems include, for example, streptomycete bacteriophage phiC31 (Kuhstoss et al. (1991) J Mol Biol 20:897-908); bacteriophage λ (Landy (1989) Ann Rev Biochem 58:913-949, and Landy (1993) Curr Op Genet Dev 3:699-707); SSV1 site-specific recombination system from Sulfolobus shibatae (Maskhelishvili et al. (1993) Mol Gen Genet 237:334-342); and a retroviral integrase-based integration system (Tanaka et al. (1998) Gene 17:67-76). In some examples, the recombinase is one that does not require cofactors or a supercoiled substrate. Such recombinases include Cre, FLP, phiC31 Int, mutant λ Int, or active variants or fragments thereof. FLP recombinase catalyzes a site-specific reaction between two FRT sites, and is involved in amplifying the copy number of the two-micron plasmid of S. cerevisiae during DNA replication. The FLP protein has been cloned and expressed. See, for example, Cox (1993) Proc Natl Acad Sci USA 80:4223-4227. The FLP recombinase used may be derived from the genus Saccharomyces. In some examples a polynucleotide encoding the recombinase synthesized using plant-preferred codons is used. FLP enzyme encoded by a polynucleotide comprising maize preferred codons (FLPm) that catalyzes site-specific recombination events is known (U.S. Pat. No. 5,929,301). Additional functional variants and fragments of FLP are known. See, for example, Buchholz et al. (1998) Nat Biotechnol 16:617-618, Hartung et al. (1998) J Biol Chem 273:22884-22891, Saxena et al. (1997) Biochim Biophys Acta 1340:187-204, Hartley et al. (1980) Nature 286:860-864, Shaikh & Sadowski (2000) J Mol Biol 302:27-48, Voziyanov et al. (2002) Nucleic Acids Res 30:1656-1663, and Voziyanov et al. (2003) J Mol Biol 326:65-76. The bacteriophage P1 recombinase Cre catalyzes site-specific recombination between two lox sites. See, for example, Guo et al. (1997) Nature 389:40-46; Abremski et al. (1984) J Biol Chem 259:1509-1514; Chen et al. (1996) Somat Cell Mol Genet 22:477-488; Shaikh et al. (1977) J Biol Chem 272:5695-5702; and Buchholz etal. (1998) Nat Biotechnol 16:617-618. Cre polynucleotide sequences may also be synthesized using plant-preferred codons, for example, moCre (see, e.g., WO 99/25840), and other variants are known, see for example Vergunst et al. (2000) Science 290:979-982, Santoro & Schulz (2002) Proc Natl Acad Sci USA 99:4185-4190, Shaikh & Sadowski (2000) J Mol Biol 302:27-48, Rufer & Sauer (2002) Nucleic Acids Res 30:2764-2771, Wierzbicki et al. (1987) Mol Biol 195:785-794, Petyuk et al. (2004) J Biol Chem 279:37040-37048, Hartung & Kisters-Wolke (1998) J Biol Chem 273:22884-22891, Koresawa et al. (2000) J Biochem (Tokyo) 127:367-372, U.S. Pat. No. 6,890,726, and Buchholz & Stewart (2001) Nat Biotechnol 19:1047-1052. The phiC31 integrase and variants are known (Kushtoss et al. (1991) J Mol Biol 222:897-908, WO03/066867, WO05/017170, US2005/0003540, and Sclimenti et al. (2001) Nucleic Acids Res 29:5044-5051). The λ integrase and cofactors (Hoess et al. (1980) Proc Natl Acad Sci USA 77:2482-2486, Blattner etal. (1997) Science 277:1453-1474), and variants thereof are known, including cofactor-independent Int variants (Miller et al. (1980) Cell 20:721-729, Lange-Gustafson and Nash (1984) J Biol Chem 259:12724-12732, Christ et al. (1998) J Mol Biol 288:825-836, and Lorbach et al. (2000) J Mol Biol 296:1175-1181), att site recognition variants (Dorgai etal. (1995) J Mol Biol 252:178-188, Yagu et al. (1995) J Mol Biol 252:163-167, and Dorgai etal. (1998) J Mol Biol 277:1059-1070), as well as maize codon optimized Int, variant, and cofactor sequences (WO03/08045). Other integrases and variants are known, such as HK022 integrase (Kolot et al. (1999) Mol Biol Rep 26:207-213) and variants such as att site recognition variants (Dorgai et al. (1995) J Mol Biol 252:178-188, Yagu et al. (1995) J Mol Biol 252:163-167, and Dorgai etal. (1998) J Mol Biol 277:1059-1070).

Wild-type recombination sites, mutant sites, or any combination of wild type and/or mutant sites can be used. Mutant sites refer to any biologically active variant or modification of a wild type site including nucleotide substitution variants in any region, and fragments of a full-length wild type site. Such recombination sites include, for example, wild type lox, FRT, and att sites, and mutant lox, FRT, and att sites. An analysis of the recombination activity of mutant lox sites is presented in Lee et al. (1998) Gene 216:55-65. Other recombination sites and variants are known, see for example, Hoess et al. (1982) Proc Natl Acad Sci USA 79:3398-3402; Hoess et al. (1986) Nucleic Acids Res 14:2287-2300; Thomson et al. (2003) Genesis 36:162-167; Schlake & Bode (1994) Biochemistry 33:12746-12751; Siebler & Bode (1997) Biochemistry 36:1740-1747; Huang etal. (1991) Nucleic Acids Res 19:443-448; Sadowski (1995) in Progress in Nucleic Acid Research and Molecular Biology Vol. 51, pp. 53-91; Cox (1989) in Mobile DNA, Berg & Howe (eds) American Society of Microbiology, Washington D.C., pp.116-670; Dixon et al. (1995) Mol Microbiol 18:449-458; Umlauf & Cox (1988) EMBO J 7:1845-1852; Buchholz etal. (1996) Nucleic Acids Res 24:3118-3119; Kilby et al. (1993) Trends Genet 9:413-421; Rossant & Geagy (1995) Nat Med 1:592-594; Bayley et al. (1992) Plant Mol Biol 18:353-361; Odell et al. (1990) Mol Gen Genet 223:369-378; Dale & Ow (1991) Proc Natl Acad Sci USA 88:10558-10562; Qui et al. (1994) Proc Natl Acad Sci USA 91:1706-1710; Stuurman et al. (1996) Plant Mol Biol 32:901-913; Dale etal. (1990) Gene 91:79-85; Albert et al. (1995) Plant J 7:649-659, U.S. Pat. No. 6,465,254, WO01/23545, WO99/55851, and WO01/11058. In some examples, sets of dissimilar and corresponding recombination sites can be used, for example, sites from different recombination systems such as a set comprising a wild type FRT site and wild type loxP site. Accordingly, any suitable recombination site or set of recombination sites may be used, including a FRT site, a biologically active variant of a FRT site, a lox site, a biologically active variant of a lox site, an att site, a biologically active variant of an att site, any combination thereof, or any other combination of recombination sites. Examples of FRT sites include, for example, the minimal wild type FRT site (FRT1), and various mutant FRT sites, including but not limited to FRT5, FRT6, and FRT7 (see U.S. Pat. No. 6,187,994). Additional variant FRT sites are known, (see, e.g., WO 01/23545, and U.S. Provisional Ser. No. 60/700,225, filed Jul. 18, 2005, herein incorporated by reference). Other recombination sites that can be used include att sites and variants, such as those disclosed in Landy (1989) Ann Rev Biochem 58:913-949, Landy (1993) Curr Op Genet Dev 3:699-707, U.S. Pat. No. 5,888,732, WO01/07572, and Thygarajan et al. (2001) Mol Cell Biol 21:3926-3934; and lox sites and variants (see, e.g., Albert et al. 1995 Plant J 7:649-659, Hoess et al. 1986 Nucl Acids Res 14:2287-2300, Lee & Saito 1998 Gene 216:55-65, U.S. Pat. No. 6,465,254, and WO01/11058). The site-specific recombinase(s) used depend on the recombination sites in the target site and the transfer cassette. If FRT sites are utilized, FLP recombinase is provided, when lox sites are utilized, Cre recombinase is provided, when λ att sites are used, λ Int is provided, when phiC31 att sites are used, phiC31 Int is provided. If the recombination sites used comprise sites from different systems, for example a FRT and a lox site, both recombinase activities can be provided, either as separate entities, or as a chimeric recombinase, for example FLP/Cre (see, e.g., WO 99/25840).

A marker provides for the identification and/or selection of a cell, plant, and/or seed expressing the marker. Markers include, e.g., screenable, visual, and/or selectable markers. A selection marker is any marker, which when expressed at a sufficient level, confers resistance to a selective agent. Selection markers and their corresponding selective agents include, but are not limited to, herbicide resistance genes and herbicides; antibiotic resistance genes and antibiotics; and other chemical resistance genes with their corresponding chemical agents. Bacterial drug resistance genes include, but are not limited to, neomycin phosphotransferase II (nptll) which confers resistance to kanamycin, paromycin, neomycin, and G418, and hygromycin phosphotransferase (hph) which confers resistance to hygromycin B. See also, Bowen (1993) Markers for Plant Gene Transfer, Transgenic Plants, Vol.1, Engineering and Utilization; Everett etal. (1987) Bio/Technology 5:1201-1204; Bidney et al. (1992) Plant Mol Biol 18:301-313; and WO97/05829. Resistance may also be conferred to herbicides from several groups, including amino acid synthesis inhibitors, photosynthesis inhibitors, lipid inhibitors, growth regulators, cell membrane disrupters, pigment inhibitors, seedling growth inhibitors, including but not limited to imidazolinones, sulfonylureas, triazolopyrimidines, glyphosate, sethoxydim, fenoxaprop, glufosinate, phosphinothricin, triazines, bromoxynil, and the like. See, for example, Holt (1993) Ann Rev Plant Physiol Plant Mol Biol 44:203-229; and Miki et al. (2004) J Biotechnol 107:193-232. Selection markers include sequences that confer resistance to such herbicides and include, but are not limited to, the bar gene, which encodes phosphinothricin acetyl transferase (PAT) which confers resistance to glufosinate (Thompson et al. (1987) EMBO J 6:2519-2523); glyphosate oxidoreductase (GOX), glyphosate N-acetyltransferase (GAT), and 5-enol pyruvylshikimate-3-phosphate synthase (EPSPS) each of which confers resistance to glyphosate (Barry et al. (1992) in Biosynthesis and Molecular Regulation of Amino Acids in Plants, B. K. Singh etal. (Eds) pp.139-145; Kishore et al. (1992) Weed Tech 6:626-634; Castle (2004) Science 304:1151-1154; Zhou et al. (1995) Plant Cell Rep 15:159-163; WO97/04103; WO02/36782; and WO03/092360). Other selection markers include dihydrofolate reductase (DHFR), which confers resistance to methotrexate (see, e.g., Dhir et al. (1994) Improvements of Cereal Quality by Genetic Engineering, R. J. Henry (ed), Plenum Press, New York; and Hauptmann et al. (1988) Plant Physiol 86:602-606). Acetohydroxy acid synthase (AHAS orALS) mutant sequences lead to resistance to imidiazolinones and/or sulfonylureas such as imazethapyr and/or chlorsulfuron (see, e.g., Zu et al. (2000) Nat Biotechnol 18:555-558; U.S. Pat. Nos. 6,444,875, and 6,660,910; Sathasivan et al. (1991) Plant Physiol 97:1044-1050; Ott et al. (1996) J Mol Biol 263:359-368; and Fang et al. (1992) Plant Mol Biol 18:1185-1187). In addition, chemical resistance genes further include tryptophan decarboxylase which confers resistance to 4-methyl tryptophan (4-mT) (Goodijn et al. (1993) Plant Mol Biol 22:907-912); and bromoxynil nitrilase which confers resistance to bromoxynil. The selection marker may comprise cyanamide hydratase (Cah), see, for example, Greiner et al. (1991) Proc Natl Acad Sci USA 88:4260-4264; and Weeks etal. (2000) Crop Sci 40:1749-1754. Cyanamide hydratase enzyme converts cyanamide into urea, thereby conferring resistance to cyanamide. Any form or derivative of cyanamide can be used as a selection agent including, but not limited to, calcium cyanamide (Perlka® (SKW, Trotberg Germany) and hydrogen cyanamide (Dormex® (SKW)). See also, U.S. Pat. Nos. 6,096,947, and 6,268,547. Variants of cyanamide hydratase polynucleotides and/or polypeptides will retain cyanamide hydratase activity. A biologically active variant of cyanamide hydratase will retain the ability to convert cyanamide to urea. Methods to assay for such activity include assaying for the resistance of plants expressing the cyanamide hydratase to cyanamide. Additional assays include the cyanamide hydratase colorimetric assay (see, e.g., Weeks etal. (2000) Crop Sci 40:1749-1754; and U.S. Pat. No. 6,268,547).

A polynucleotide indicates any nucleic acid molecule, and comprises naturally occurring, synthetic, and/or modified ribonucleotides, deoxyribonucleotides, and combinations of ribonucleotides and deoxyribonucleotides. Polynucleotides encompass all forms of sequences including, but not limited to, single-stranded, double-stranded, linear, circular, branched, hairpins, stem-loop structures, and the like. A DNA construct comprises a polynucleotide which, when present in the genome of a plant, is heterologous or foreign to that chromosomal location in the plant genome. In preparing the DNA construct, various fragments may be manipulated to provide the sequences in a proper orientation and/or in the proper reading frame. Adapters or linkers may be employed to join the fragments. Other manipulations may be used to provide convenient restriction sites, removal of superfluous DNA, or removal of restriction sites. For example, in vitro mutagenesis, primer repair, restriction, annealing, resubstitutions, transitions, transversions, or recombination systems may be used. Polynucleotides, including polynucleotides of interest, markers, polynucleotides encoding a recombinase, recombination sites, target sites, transfer cassettes, can be provided in a DNA construct. The construct can include 5′ and 3′ regulatory sequences operably linked to the appropriate sequences. The DNA construct can include in the 5′ to 3′ direction of transcription at least one of the following, a transcriptional initiation region, a translational initiation region, the polynucleotide, and a transcriptional and/or translational termination region functional in plants. Alternatively, the DNA construct may lack at least one 5′ and/or 3′ regulatory element. For example, a DNA construct may be designed such that upon introduction into a cell and in the presence of the appropriate recombinase a recombination event at the target site operably links the 5′ and/or 3′ regulatory regions to the appropriate sequences of the DNA construct. Operably linked means that the nucleic acid sequences linked are contiguous and comprise a functional linkage of the components. Regulatory elements can be used in a variety of ways depending on the polynucleotide element, recombination site, transfer cassette and/or target site employed. In some examples intervening sequences can be present between operably linked elements and not disrupt the functional linkage. For example, an operable linkage between a promoter and a polynucleotide of interest allows the promoter to initiate and mediate transcription of the polynucleotide of interest. In some examples an ATG start codon is operably linked to a recombination site. In some examples, a recombination site is within an intron. The cassette may additionally contain at least one additional sequence to be introduced into the plant. Alternatively, additional sequence(s) can be provided separately. A DNA construct can be provided with a plurality of restriction sites and/or recombination sites for manipulation of the various components and elements. The DNA cassette may additionally contain selectable marker genes.

A transcriptional initiation region may be native, analogous, foreign, or heterologous to the plant host or to the polynucleotide of interest, and may be a natural sequence, a modified sequence, or a synthetic sequence. A number of promoters can be used to express a coding sequence. A variety of promoters useful in plants is reviewed in Potenza et al. (2004) In Vitro Cell Dev Biol Plant 40:1-22. In some examples, the promoter expressing the selection marker is active in the seed. Promoters active in the seed include constitutive promoters, developmental promoters, tissue specific promoters, inducible promoters, and the like. Constitutive promoters include for example, the core promoter of the Rsyn7 promoter and other constitutive promoters disclosed in WO99/43838 and U.S. Pat. No. 6,072,050; the core CaMV 35S promoter (Odell et al. (1985) Nature 313:810-812); the MVV (mirabilis mosaic virus) promoter (Dey & Maiti (1999) Plant Mol Biol 40:771-782); rice actin (McElroy et al. (1990) Plant Cell 2:163-171); ubiquitin (Christensen et al. (1989) Plant Mol Biol 12:619-632, and Christensen etal. (1992) Plant Mol Biol 18:675-689); PEMU (Last et al. (1991) Theor Appl Genet 81:581-588); MAS (Velten et al. (1984) EMBO J 3:2723-2730); ALS promoter (U.S. Pat. No. 5,659,026), and the like. Other constitutive promoters include those disclosed in, e.g., U.S. Pat. Nos. 5,608,149; 5,608,144; 5,604,121; 5,569,597; 5,466,785; 5,399,680; 5,268,463; 5,608,142; and 6,177,611. The promoter may be a tissue-preferred promoter, e.g. to target enhanced expression within a particular plant tissue. In some examples, a seed-preferred promoter is used to express the selection marker. Seed-preferred promoters include both seed-specific promoters, active during seed development, as well as seed-germinating promoters, active during seed germination. See Thompson et al. (1989) BioEssays 10:108. Seed-preferred promoters include, but are not limited to, cim1 (cytokinin-induced message); jip1 (jasmonate-induced protein) (see WO02/42424); cZ19B1 (maize 19 kDa zein); mi1ps, milps3 (myo-inositol-1-phosphate synthase) (see WO0/11 1177, WO02/42424, and U.S. Pat. No. 6,225,529); lec1 (see WO02/42424); oleosin (see Qu & Huang (1990) J Biol Chem 265:2238-2243, and Plant et al. (1994) Plant Mol Biol 25:193-202); rab17 (see Vilardell et al. 1990 Plant Mol Biol 14:423-432; Vilardell et al. 1991 Plant Mol Biol 17:985-993; and Busk et al. 1997 Plant J 11:1285-1295); bean β-phaseolin; napin; β-conglycinin; soybean lectin; cruciferin; maize 15 kDa zein; 22 kDa zein; 27 kDa zein; waxy; shrunken1, shrunken2; globulin1, globulin2; end1, and end2 (WO00/12733); and the like. In other examples, a chemical-regulated promoter is used. A chemical-regulated promoter can be used to modulate expression in the seed through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzenesulfonamide herbicide safeners; the maize GST promoter, activated by hydrophobic electrophilic compounds (e.g., some pre-emergent herbicides); and the tobacco PR-1 a promoter, activated by salicylic acid. Other chemical-regulated promoters of interest include steroid-responsive promoters (see, for example, the glucocorticoid-inducible promoter in Schena et al. (1991) Proc Natl Acad Sci USA 88:10421-10425; and McNellis et al. (1998) Plant J 14:247-257) and tetracycline-inducible and tetracycline-repressible promoters (see, e.g., Gatz et al. (1991) Mol Gen Genet 227:229-237, and U.S. Pat. Nos. 5,814,618, and 5,789,156).

The DNA construct can comprise expression units. An expression unit comprises a promoter operably linked to another polynucleotide sequence. Expression units can have additional elements including, but not limited to, introns, enhancers, leaders insulators, polynucleotides of interest, marker genes, recombination sites, translation initiation regions, termination regions, sequences encoding recombinases, etc. In addition, the DNA constructs can comprise transfer cassettes, target sites, or any portions or combinations thereof. The DNA construct can be modified in a variety of ways, including site-specific recombination methods, to provide a number of variations in the DNA construct. Polynucleotide sequences may be modified for expression in the plant. See, e.g., Campbell & Gowri (1990) Plant Physiol 92:1-11. Methods for synthesizing plant-preferred genes include, e.g., U.S. Pat. Nos. 5,380,831, 5,436,391, and Murray et al. (1989) Nucleic Acids Res 17:477-498. Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to average levels for a given host, as calculated by reference to endogenous genes expressed in the host. The sequence may also be modified to avoid secondary mRNA structures. Cassettes may additionally contain 5′ leader sequences in the DNA cassette which may act to enhance translation. Translation leaders include, e.g., picornavirus leaders such as EMCV leader (Elroy-Stein et al. (1989) Proc Natl Acad Sci USA 86:6126-6130); potyvirus leaders such as TEV leader (Gallie et al. (1995) Gene 165:233-238), MDMV leader (Kong et al. (1988) Arch Virol 143:1791-1799), and human immunoglobulin heavy-chain binding protein (BiP) (Macejak et al. (1991) Nature 353:9094); untranslated leader from the coat protein mRNA of alfalfa mosaic virus (AMV RNA 4) (Jobling et al. (1987) Nature 325:622-6255); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol 84:965-968. Other methods or sequences known to enhance translation can also be utilized, such as introns, and the like.

Polynucleotides of interest include, e.g., zinc fingers, kinases, heat shock proteins, transcription factors, DNA repair factors, agronomic traits, insect resistance, disease resistance, herbicide resistance, sterility, oil, protein, starch, digestibility, kernel size, maturity, nutrient composition, levels or metabolism, and the like. Insect resistance genes may encode resistance to pests such as rootworm, cutworm, European Corn Borer, and the like. Such genes include, e.g., B. thuringiensis toxic protein genes (U.S. Pat. Nos. 5,366,892; 5,747,450; 5,736,514; 5,723,756; 5,593,881; Geiser et al. (1986) Gene 48:109); and the like. Disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Herbicide resistance traits include genes coding for resistance to herbicides including sulfonylurea-type herbicides (e.g., the S4 and/or Hra mutations in ALS), herbicides that act to inhibit action of glutamine synthase, such as phosphinothricin or basta (e.g., the bar gene), EPSPS (U.S. Pat. Nos. 6,867,293; 5,188,642; and 5,627,061), GOX (Zhou etal. (1995) Plant Cell Rep 15:159-163), and GAT (U.S. Pat. No. 6,395,485). Antibiotic resistance genes may also be used, such as the nptil gene which encodes resistance to the antibiotics kanamycin and geneticin. Sterility genes can also be used, for example as an alternative to detasseling, including male tissue-preferred genes and genes with male sterility phenotypes such as QM (e.g., U.S. Pat. No. 5,583,210), kinases, and those encoding compounds toxic to either male or female gametophytic development.

Reduction of the activity of specific genes, silencing and/or suppression may be desired. Many techniques for gene silencing are known, including but not limited to antisense technology (see, e.g., Sheehy et al. (1988) Proc Natl Acad Sci USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech 8:340-344; Flavell (1994) Proc Natl Acad Sci USA 91:3490-3496; Finnegan et al. (1994) Bio/Technology 12:883-888; and Neuhuber et al. (1994) Mol Gen Genet 244:230-241); RNA interference (Napoli et al. (1990) Plant Cell 2:279-289; U.S. Pat. No. 5,034,323; Sharp (1999) Genes Dev 13:139-141; Zamore etal. (2000) Cell 101:25-33; Javier (2003) Nature 425:257-263; and, Montgomery et al. (1998) Proc Natl Acad Sci USA 95:15502-15507), virus-induced gene silencing (Burton et al. (2000) Plant Cell 12:691-705; and Baulcombe (1999) Curr Op Plant Bio 2:109-113); target-RNA-specific ribozymes (Haseloff et al. (1988) Nature 334: 585-591); hairpin structures (Smith et al. (2000) Nature 407:319-320; WO99/53050; WO2/00904; and WO98/53083); ribozymes (Steinecke et al. (1992) EMBO J 11:1525; U.S. Pat. No. 4,987,071; and, Perriman et al. (1993) Antisense Res Dev 3:253); oligonucleotide mediated targeted modification (e.g., WO03/076574: and WO99/25853); Zn-finger targeted molecules (e.g., WO01/52620; WO03/048345; and WO0/42219); and other methods, or combinations of the above methods.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol Gen Genet 262:141-144; Proudfoot (1991) Cell 64:671-674; Sanfacon et al. (1991) Genes Dev 5:141-149; Mogen et al. (1990) Plant Cell 2:1261-1272; Munroe et al. (1990) Gene 91:151-158; Ballas etal. (1989) Nucleic Acids Res 17:7891-7903; and Joshi etal. (1987) Nucleic Acids Res 15:9627-9639.

Any method for introducing a sequence into a plant can be used, as long as the polynucleotide or polypeptide gains access to the interior of at least one cell. Methods for introducing sequences into plants are known and include, but are not limited to, stable transformation, transient transformation, virus-mediated methods, and sexual breeding. Stably incorporated indicates that the introduced polynucleotide is integrated into a genome and is capable of being inherited by progeny. Transient transformation indicates that an introduced sequence does not integrate into a genome such that it is heritable by progeny from the host. The plants and seeds employed may have a DNA construct stably incorporated into their genome. Any protocol may be used to introduce the DNA construct, any component of site-specific recombination systems, a polypeptide, or any other polynucleotide of interest. Providing comprises any method that brings together any polypeptide and/or a polynucleotide with any other recited component(s). Any means can be used to bring together a target site, transfer cassette, and appropriate recombinase, including, for example, stable transformation, transient delivery, and sexual crossing (see, e.g., WO99/25884). In some examples, the recombinase may be provided in the form of the polypeptide or mRNA. A series of protocols may be used in order to bring together the various components. For instance, a cell can be provided with at least one of these components via a variety of methods including transient and stable transformation methods; co-introducing a recombinase DNA, mRNA or protein directly into the cell; employing an organism (e.g., a strain or line) that expresses the recombinase; or growing/culturing the cell or organism carrying a target site, crossing to an organism expressing an active recombinase protein, and selecting events in the progeny. A simple integration pattern is produced when the transfer cassette integrates predominantly at the target site, and at less than about 25, 20,15, 10, 9, 8, 7, 6, 5, 4, 3, 2, or 1 random position(s) in the genome. Any promoter, including constitutive, inducible, developmentally, temporal, and/or spatially regulated promoter, etc., that is capable of regulating expression in the organism may be used.

Transformation protocols as well as protocols for introducing polypeptides or polynucleotide sequences into plants may vary depending on the type of plant or plant cell targeted for transformation. Suitable methods of introducing polypeptides and polynucleotides into plant cells include microinjection (Crossway etal. (1986) Biotechniques 4:320-334, and U.S. Pat. No. 6,300,543), electroporation (Riggs et al. (1986) Proc Natl Acad Sci USA 83:5602-5606, Agrobacterium-mediated transformation (U.S. Pat. Nos. 5,563,055; and 5,981,840), direct gene transfer (Paszkowski et al. (1984) EMBO J 3:2717-2722), and ballistic particle acceleration (U.S. Pat. Nos. 4,945,050; 5,879,918; 5,886,244; and 5,932,782; Tomes et al. (1995) in Plant Cell, Tissue, and Organ Culture: Fundamental Methods, ed. Gamborg & Phillips (Springer-Verlag, Berlin); McCabe et al. (1988) Biotechnology 6:923-926); and Lec1 transformation (WO00/28058). Also see Weissinger et al. (1988) Ann Rev Genet 22:421-477; Sanford et al. (1987) Particulate Science and Technology 5:27-37 (onion); Christou et al. (1988) Plant Physiol 87:671-674 (soybean); Finer & McMullen (1991) In Vitro Cell Dev Biol 27P:175-182 (soybean); Singh etal. (1998) TheorAppl Genet 96:319-324 (soybean); Datta etal. (1990) Biotechnology 8:736-740 (rice); Klein et al. (1988) Proc Natl Acad Sci USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) Proc Natl Acad Sci USA 84:5345-5349 (Liliaceae); De Wet et al. (1985) in The Experimental Manipulation of Ovule Tissues, ed. Chapman et al. (Longman, New York), pp.197-209 (pollen); Kaeppler et al. (1990) Plant Cell Rep 9:415-418; and Kaeppler et al. (1992) Theor Appl Genet 84:560-566 (whisker-mediated transformation); D'Halluin et al. (1992) Plant Cell 4:1495-1505 (electroporation); Li et al. (1993) Plant Cell Rep 12:250-255; Christou & Ford (1995) Ann Bot 75:407-413 (rice); and Osjoda et al. (1996) Nat Biotechnol 14:745-750 (maize via A. tumefaciens).

The polynucleotide may be introduced into plants by contacting plants with a virus, or viral nucleic acids. Generally, such methods involve incorporating a desired polynucleotide within a viral DNA or RNA molecule. The sequence may initially be synthesized in a viral polyprotein and later processed in vivo or in vitro to produce a desired protein. Useful promoters encompass promoters utilized for transcription by viral RNA polymerases. Methods for introducing polynucleotides into plants and expressing a protein encoded, involving viral DNA or RNA molecules, are known, see, e.g., U.S. Pat. Nos. 5,889,191; 5,889,190; 5,866,785; 5,589,367; 5,316,931; and Porta et al. (1996) Mol Biotech 5:209-221.

Various components, including those from a site-specific recombination system, can be provided to a plant using a variety of transient methods. Such transient transformation methods include, but are not limited to, providing recombinase polypeptide directly, providing a recombinase mRNA, using a non-integrative method, or providing low levels of DNA into the plant. Such methods include, for example, microinjection, particle bombardment, viral vector systems, and/or precipitation of the polynucleotide wherein transcription occurs from the particle-bound DNA without substantive release or integration into the genome, such methods generally use particles coated with polyethylimine, (see, e.g., Crossway et al. (1986) Mol Gen Genet 202:179-185; Nomura et al. (1986) Plant Sci 44:53-58; Hepler et al. (1994) Proc Natl Acad Sci USA 91:2176-2180; and Hush et al. (1994) J Cell Sci 107:775-784).

The term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included.

Any plant species can be used with the methods and compositions, including, but not limited to, monocots and dicots. Examples of plant genuses and species include, but are not limited to, corn (Zea mays), Brassica sp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses and conifers. Vegetables include tomatoes (Lycopersicon esculentum), lettuce (e.g., Lactuca sativa), green beans (Phaseolus vulgaris), lima beans (Phaseolus limensis), peas (Pisium spp., Lathyrus spp.), and Cucumis species such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include pines, for example, loblolly pine (Pinus taeda), slash pine (Pinus elliotii), ponderosa pine (Pinus ponderosa), lodgepole pine (Pinus contorta), and Monterey pine (Pinus radiata), Douglas fir (Pseudotsuga menziesii); Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicata) and Alaska yellow cedar (Chamaecyparis nootkatensis).

The cells that have been transformed may be grown into plants in accordance with conventional ways. See, for example, McCormick et al. (1986) Plant Cell Rep 5:81-84. These plants may then be grown and self-pollinated, backcrossed, and/or outcrossed, and the resulting progeny having the desired characteristic identified. Two or more generations may be grown to ensure that the characteristic is stably maintained and inherited and then seeds harvested. In this manner transformed/transgenic seed having the recited DNA construct stably incorporated into their genome are provided. A plant and/or a seed having stably incorporated the DNA construct can be further characterized for expression, site-specific integration potential, agronomics, and copy number (see, e.g., U.S. Pat. No. 6,187,994).

Sequence identity may be used to compare the primary structure of two polynucleotides or polypeptide sequences. Sequence identity measures the residues in the two sequences that are the same when aligned for maximum correspondence. Sequence relationships can be analyzed using computer-implemented algorithms. The sequence relationship between two or more polynucleotides, or two or more polypeptides can be determined by computing the best alignment of the sequences, and scoring the matches and the gaps in the alignment, which yields the percent sequence identity, and the percent sequence similarity. Polynucleotide relationships can also be described based on a comparison of the polypeptides each encodes. Many programs and algorithms for comparison and analysis of sequences are known. Unless otherwise stated, sequence identity/similarity values provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff & Henikoff (1992) Proc. Natl. Acad. Sci USA 89:10915-10919). GAP uses the algorithm of Needleman & Wunsch (1970) J Mol Biol 48:443-453, to find the alignment of two complete sequences that maximizes the number of matches and minimizes the number of gaps.

Variant polynucleotides include polynucleotides having at least one deletion, addition, and/or substitution in at least one of the 5′ end, 3′ end, and/or internal sites including introns or exons, as compared to the native polynucleotide. Variant polynucleotides include naturally occurring variants as well as synthetically derived polynucleotides, for example, those generated using site-directed mutagenesis. Conservative variants include sequences that maintain their function, encode the same polypeptide, or encode a variant polypeptide with substantially similar identity, function, and/or activity as the native polynucleotide. Variants can be identified with known techniques, for example, polymerase chain reaction (PCR), and/or hybridization techniques. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide. Variant polynucleotides can also be evaluated by comparison of the percent sequence identity between the polypeptides encoded using standard alignment programs and parameters. When evaluated by comparison of the percent sequence identity shared by the polypeptides each encodes, the percent sequence identity between the two encoded polypeptides is typically at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

Variant proteins include proteins having at least one deletion, addition, and/or substitution in at least one of the N-terminal end, C-terminal end, and/or an internal site, as compared to the native polypeptide. Variant proteins possess the desired biological activity of the protein. Variants include naturally occurring polypeptides, as well as those generated by human manipulation. Biologically active variants of a protein typically have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs. A biologically active variant of a protein may differ from that protein by as few as 1-15 amino acid residues. Conservative substitutions generally refer to exchanging one amino acid with another having similar properties. For example, the model of Dayhoff et al. (1978) Atlas of Protein Sequence and Structure (Natl Biomed Res Found, Washington, D.C.) provides guidance on amino acid substitutions that are not expected to affect the biological activity of the protein.

Variant polynucleotides and proteins encompass sequences derived from mutagenic and/or recombinogenic procedures, such as mutagenesis and/or DNA shuffling. Methods for mutagenesis, nucleotide sequence alterations, and DNA shuffling are known (see, e.g., Kunkel (1985) Proc Natl Acad Sci USA 82:488-492; Kunkel et al. (1987) Methods Enzymol 154:367-382; U.S. Pat. No. 4,873,192; Walker & Gaastra, eds. (1983) Techniques in Molecular Biology (MacMillan Publ. Co., N.Y.); Stemmer (1994) Proc Natl Acad Sci USA 91:10747-10751; Stemmer (1994) Nature 370:389-391; Crameri et al. (1997) Nat Biotechnol 15:436-438; Moore et al. (1997) J Mol Biol 272:336-347; Zhang et al. (1997) Proc Natl Acad Sci USA 94:4504-4509; Crameri et al. (1998) Nature 391:288-291; and U.S. Pat. Nos. 5,605,793, and 5,837,458). For example, one or more different recombinase coding sequences can be manipulated to create and select a new recombinase protein possessing the desired properties. DNA shuffling typically generates libraries of recombinant polynucleotides from a population of related sequences, which are homologously recombined in vitro or in vivo. Generally, any modification(s) to a polynucleotide encoding a polypeptide should not alter the reading frame, or create and/or alter DNA or mRNA secondary structure. See, EP Patent Application Publication No. 75,444 .

Fragments and variants of recombination sites, recombinases, selection markers, and nucleotide sequences of interest can be used, and unless otherwise stated, indicate that the variant or fragment retains at least some of the activity/function of the original composition. For example, for a polynucleotide encoding a protein, a fragment of a polynucleotide encodes a polypeptide protein that retains at least some of the biological activity of the full-length protein. Fragments of a polynucleotide may range from at least about 20 nucleotides, about 50 nucleotides, about 100 nucleotides, up to the full-length polynucleotide. A fragment of a polynucleotide that encodes a biologically active portion of a protein typically encodes at least 15, 25, 30, 50, 100, 150, 200, 250, 300, 325, 350, 375, 400, 420, or 450 contiguous amino acids, or any integer in this range up to and including the total number of amino acids present in a full-length protein. A biologically active fragment of a polypeptide can be prepared by isolating a portion of one of the polynucleotides encoding the portion of the polypeptide of interest, expressing the protein fragment, and assessing the activity. Alternatively, a biologically active fragment of a polypeptide can be produced by selective chemical or proteolytic cleavage of the full- length polypeptide, and the activity measured. For example, polynucleotides that encode fragments of a recombinase polypeptide may comprise at least 16, 20, 50, 75, 100, 150, 200, 250, 300, 350, 400, 450, 500, 550, 600, 650, 700, 800, 900, 1,000, 1,100, 1,200, 1,300, or 1,400 nucleotides, or any integer in this range up to and including the total number of nucleotides of a full-length polynucleotide. In addition, fragments of a recombination site retain the biological activity of the recombination site, undergoing a recombination event in the presence of the appropriate recombinase. Fragments of a recombination site may range from at least about 5, 10,15, 20, 25, 30, 35, 40 nucleotides, up to the full-length of a recombination site. For example, a full-length FRT, lox, attB, and attP sites are known and range from about 50 nucleotides to about 250 nucleotides, and fully active minimal sites are known and range from about 20, 25, 30, 35, 40, 45, and 50 nucleotides. Biologically active variants of a recombination site retain the biological activity of the recombination site, undergoing a recombination event in the presence of the appropriate recombinase. Biologically active variants of recombination sites have at least one substitution, addition, or deletion as compared to a full-length recombination site.

Assays to measure the biological activity of recombination sites and recombinases are known (see, e.g., Senecoll et al. (1988) J Mol Biol 201:406-421; Voziyanov et al. (2002) Nucleic Acids Res 30:7; U.S. Pat. No. 6,187,994; WO01/00158; Albert et al. (1995) Plant J 7:649-659; Hartang et al. (1998) J Biol Chem 273:22884-22891; Saxena et al. (1997) Biochim Biophy Acta 1340:187-204; and Hartley et al. (1980) Nature 280-860-864). Assays for recombinase activity measure the overall activity of the enzyme on DNA substrates containing recombination sites. For example, to assay for FLP activity, inversion of a DNA sequence in a circular plasmid containing two inverted FRT sites can be detected as a change in position of restriction enzyme sites (see, e.g., Vetter et al. (1983) Proc Natl Acad Sci USA 80:7284). Alternatively, excision of DNA from a linear molecule or intermolecular recombination frequency induced by the enzyme may be assayed (see, e.g., Babineau et al. (1985) J Biol Chem 260:12313; Meyer-Leon et al. (1987) Nucleic Acids Res 15:6469; and Gronostajski etal. (1985)J Biol Chem 260:12328). Recombinase activity may also be measured by excision of a sequence flanked by recombinogenic FRT sites to activate an assayable marker gene. Similar assays are used to measure the activity of recombination sites.

The following examples are offered by way of illustration and not by way of limitation.

### EXAMPLE 1

### Method of seed priming with Dormex®

For initial determination of the parameters, ranges, and/or conditions for seed priming, non-transgenic seed, transgenic seed transformed with a construct comprising Ubi::moCAH produced through Agrobacterium-mediated transformation were used. In further tests, transgenic seed were used comprising a putative targeted insertion and activation of the selectable marker were generated essentially as described in Example 3.

### A. Determining Soaking Time

Non-transgenic seeds from two inbred corn lines, N46 and P38 were used to evaluate the conditions for seed priming with the selective agent. For each treatment, 24 seeds each from N46 and P38 were soaked in 10 ml of water, or 0.5%, 1%, or 5% Dormex® in water for 10 min., 30 min., 1 hr., and 2 hrs, with vacuum applied. Following the soaking period, the Dormex® solution was removed and the seeds were placed into a pollination bag. A fan was placed in front of the seed bags, and the seeds were dried for times ranging from overnight to 2 days. The dried seeds were planted into flats to germinate. Table 2 provides germination results for a test with inbred N46.

TABLE 2

| | % | Soaking | | Total | | % |
|---|---|---|---|---|---|---|
| Corn Line | Dormex ® | Time | | Seed | # Seedlings | Germination |
| N46 | 0 | 10 | min. | 24 | 24 | 100 |
| N46 | 0.5 | 10 | min. | 24 | 24 | 100 |
| N46 | 1 | 10 | min. | 24 | 23 | 100 |
| N46 | 5 | 10 | min. | 24 | 19 | 79 |
| N46 | 0 | 30 | min. | 24 | 24 | 100 |
| N46 | 0.5 | 30 | min. | 24 | 23 | 96 |
| N46 | 1 | 30 | min. | 24 | 21 | 88 |
| N46 | 5 | 30 | min. | 24 | 9 | 38 |
| N46 | 0 | 1 | hr. | 24 | 24 | 100 |
| N46 | 0.5 | 1 | hr. | 24 | 22 | 92 |
| N46 | 1 | 1 | hr. | 24 | 14 | 58 |
| N46 | 5 | 1 | hr. | 24 | 2 | 8 |
| N46 | 0 | 2 | hr. | 24 | 23 | 96 |
| N46 | 0.5 | 2 | hr. | 24 | 19 | 79 |
| N46 | 1 | 2 | hr. | 24 | 13 | 54 |
| N46 | 5 | 2 | hr. | 24 | 1 | 4 |

### B. Determining Selective Agent Concentration

1. Non-Transgenic Seed

Experiments were conducted testing ranges of Dormex® concentrations ranging from 0.1%-30% v/v. Seed from inbred maize lines N46 and P38 were used, typically with a priming time of at least 2 hours, and overnight drying post-priming. Germination results of tests of Dormex® concentration are shown in Table 3.

TABLE 3

| Corn Line | % Dormex ® | Total Seed | % Germination |
|---|---|---|---|
| P38 | 0 | 10 | 100 |
| P38 | 0.1 | 10 | 40 |
| P38 | 0.3 | 10 | 0 |
| P38 | 0.5 | 10 | 0 |
| P38 | 0.7 | 10 | 0 |
| P38 | 1 | 10 | 0 |
| N46 | 0 | 10 | 100 |
| N46 | 0.1 | 10 | 20 |
| N46 | 0.3 | 10 | 0 |
| N46 | 0.5 | 10 | 0 |
| N46 | 0.7 | 10 | 0 |
| N46 | 1 | 10 | 0 |
| N46 | 0 | 96 | 96 |
| N46 | 5 | 96 | 0 |
| N46 | 10 | 96 | 0 |
| N46 | 15 | 92 | 0 |
| N46 | 30 | 96 | 0 |

2. Transgenic Seed

A range of Dormex® concentrations and treatment times were tested, ranging from 0.5% -5% v/v. Seed from each independent transgenic event, hemizygous for ubi::moCah, were prepared by placing seed into mesh bags. The seeds were then placed into a Dormex® solution +0.4% glycerol (1 L comprised: desired volume of Dormex®, 4 ml glycerol and sterile deionized water to 1 L) to just cover seed. The seeds were incubated for 18 hours in the dark at 20° C. Following the incubation, the seeds were removed from the Dormex® solution, rinsed with water, allowed to dry for 1-3 days before germinating. Typically, optimal germination conditions were obtained by wrapping the dry seed in sterile moistened germination paper and incubation in the dark at 37° C. for 3-4 days. Germinated seedlings were further screened by PCR to confirm the presence of the moCAH gene. In the first trial, events with higher copy number tended to show lower Dormex® resistance, a possible indication of transgene rearrangement. Events with 2 or fewer copies of the transgene were re-tested. TABLE 4 presents the percentage of germinated seedlings using varying Dormex® concentrations for priming for the two combined tests. Some escapes could be found at Dormex® lower concentrations lower than 1%, as determined by PCR verification for the moCah gene.

TABLE 4

| | Dormex ® concentration | | | | | |
|---|---|---|---|---|---|---|
| Event | 0% | 0.3% | 0.5% | 0.7% | 1% | 1.5% |
| N46_2237612 | 100 | 10 | 0 | 0 | 0 | 0 |
| P38_2242574 | 100 | 80 | 0 | 0 | 0 | 0 |
| P38_2145089 | 87 | 100 | 77 | 67 | 63 | 50 |
| N46_2145082 | 70 | 30 | 37 | 43 | 23 | 27 |
| P38_2171582 | 80 | 60 | 54 | 64 | 36 | 40 |
| N46_2171578 | 87 | 93 | 63 | 93 | 60 | 73 |
| N46_2145072 | 70 | 60 | 72 | 80 | 80 | 32 |
| P38_2237625 | 90 | 10 | 0 | 0 | 0 | 0 |
| N46_1887662 | 80 | 80 | 80 | 80 | 80 | 50 |

In a separate experiment, transgenic inbred lines N46 and P38, comprising Ubi::moCah, and non-transgenic controls were primed in 0-5% v/v Dormex® solution, with no additives, for 3 hours, transferred to mesh bags, dried overnight in front of a fan, and planted in flats for germination. The presence of moCah was verified by enzyme assay &/or PCR analysis of selected germinated seedlings. The results are shown in Table 5.

TABLE 5

| | % | Total | % | | PCR |
|---|---|---|---|---|---|
| Corn Line | Dormex ® | Seed | Germination | Enzyme | moCah+ |
| N46 | 0 | 48 | 98 | 0/8 | 0 |
| N46 | 1 | 48 | 6 | 0/2 | 0 |
| N46 | 3 | 44 | 0 | — | — |
| N46 | 5 | 47 | 0 | — | — |
| N46_1422777 | 0 | 48 | 67 | — | — |
| N46_1422777 | 1 | 46 | 27 | 0/13 | — |
| N46_1422777 | 5 | 43 | 7 | 0/3 | 3/3 |
| N46_1422780 | 5 | 36 | 44 | 1/16 | 10/10 |
| N46_2025731 | 0 | 48 | 98 | — | — |
| N46_2025731 | 3 | 48 | 54 | 21/26 | — |
| N46_2025731 | 5 | 48 | 19 | 7/9 | 7/7 |
| P38_1370184 | 0 | 48 | 100 | — | — |
| P38_1370184 | 1 | 48 | 77 | 4/37 | — |
| P38_1370184 | 3 | 48 | 67 | 3/33 | — |
| P38_1370184 | 5 | 48 | 31 | 5/15 | 9/10** |
| P38_1445805 | 5 | 35 | 14 | 5/5 | — |
| P38_2171582 | 5 | 7 | 29 | 2/2 | 2/2 |
| P38 | 0 | 41 | 93 | 0/7 | 0/2 |
| P38 | 1 | 48 | 0 | — | — |
| P38 | 3 | 48 | 0 | — | — |
| P38 | 5 | 32 | 0 | — | — |

**DNA isolation failed from one sample

### C. Seed Priming Selection of Recombinant Target

Non-transgenic and transgenic maize seeds were placed in nylon net bags and soaked in 3% or 5% Dormex® solution for 3 hrs. Two lots of Dormex® were used in this experiment. The seeds were removed from the bags, placed on a flat surface and dried for 3 days, some dried primed seed were then planted into flats to germinate, and remaining primed seed were stored. Selected germinated seedlings were further analyzed for cyanamide hydratase enzyme activity, and by PCR to confirm the linkage of the ubiquitin promoter to the moCAH polynucleotide. These results are provided in Table 6. Samples N46-1 through P38-12 are non-transgenic controls, no germination was observed after priming with Dormex®. Samples N46-13 through N46-21 are transgenic seed, N46-21 is a transgenic control primed with water, N46-13 and N46-14 are from a transgenic target line comprising PHP17797, which lacks the moCAH selectable marker, the remaining samples are paired samples of targeted seed testing linkage and activation of donor moCAH selectable marker to the target ubiquitin promoter.

TABLE 6

| Corn | | | Total | | | | | |
|---|---|---|---|---|---|---|---|---|
| Line | Dormex ® | Lot | seed | Planted | Seedling | % Germination | Enzyme | PCR+ |
| N46-1 | 3% | 1 | 24 | 12 | 0 | 0 | — | — |
| N46-2 | 3% | 1 | 100 | 12 | 0 | 0 | — | — |
| N46-3 | 3% | 2 | 24 | 12 | 0 | 0 | — | — |
| N46-4 | 3% | 2 | 100 | 12 | 0 | 0 | — | — |
| N46-5 | 5% | 2 | 24 | 12 | 0 | 0 | — | — |
| N46-6 | 5% | 2 | 100 | 12 | 0 | 0 | — | — |
| P38-7 | 3% | 1 | 24 | 12 | 0 | 0 | — | — |
| P38-8 | 3% | 1 | 100 | 12 | 0 | 0 | — | — |
| P38-9 | 3% | 2 | 24 | 12 | 0 | 0 | — | — |
| P38-10 | 3% | 2 | 100 | 12 | 0 | 0 | — | — |
| P38-11 | 5% | 2 | 24 | 12 | 0 | 0 | — | — |
| P38-12 | 5% | 2 | 100 | 12 | 0 | 0 | — | — |
| N46-13 | 3% | 2 | 18 | 9 | 0 | 0 | — | — |
| N46-14 | 5% | 2 | 18 | 9 | 0 | 0 | — | — |
| P38-15 | 3% | 2 | 50 | 24 | 13 | 54 | 12/13 | 12/13 |
| P38-16 | 5% | 2 | 43 | 24 | 6 | 25 | 6/6 | 6/6 |
| N46-17 | 3% | 2 | 25 | 12 | 5 | 42 | 5/5 | 5/5 |
| N46-18 | 5% | 2 | 25 | 12 | 1 | 8 | 1/1 | 1/1 |
| N46-19 | 3% | 2 | 25 | 12 | 1 | 8 | 0/1 | 0/1 |
| N46-20 | 5% | 2 | 25 | 12 | 0 | 0 | — | — |
| N46-21 | 0% | 2 | 12 | 12 | 11 | 92 | 0/11 | 0/11 |

The data demonstrate that escapes were possible at priming times less than 3 hours. In addition, variable Dormexe tolerance was detected in the different corn genotypes tested. Specifically, it was found that N46 wild type seeds are more tolerant to Dormex® than P38 wild type seeds. It was also observed that the concentration of Dormex® effective to select events by seed priming was about 1/10 of the concentration typically used for leaf painting or spraying plants (600 mM). The concentration of selective agent and treatment time should be evaluated for seeds of different genotypes.

### D. Drying the Primed Seed

After priming, the seeds may be planted and allowed to germinate, or the primed seeds can be dried for storage. The appropriate conditions (temperature, relative humidity, and time) for the drying process will vary depending on the seed and can be determined empirically. See, for example, Jeller et al. (2003) Braz J Biol 63:61-68. Drying the primed seed includes a superficial drying of the seed or, alternatively, drying the seed back to its original water content. Alternatively, primed seed can be germinated immediately after priming without drying. Further, the dried seeds can be immediately germinated or can be stored under appropriate conditions. All of the above methods were tested and have been used. The method adopted was to dry the seed in a drying oven, or in the greenhouse, for 3 days followed by storage at a cool dry temperature for up to a month.

Similar testing can be done to determine appropriate priming conditions for this and other selective agents in other corn inbred lines, corn hybrids, and other seeds such as soybean, rice, cotton, canola, etc.

Seed from non-transgenic inbred lines N46 and P38 primed with Dormex® concentrations ranging from 0-1% v/v, in parallel with one test shown in Table 3, and germinated after 6 days of drying & storage. The germination results are shown in Table 7.

TABLE 7

| Corn Line | % Dormex ® | Total Seed | % Germination |
|---|---|---|---|
| P38 | 0 | 10 | 100 |
| P38 | 0.1 | 10 | 60 |
| P38 | 0.3 | 10 | 0 |
| P38 | 0.5 | 10 | 0 |
| P38 | 0.7 | 10 | 0 |
| P38 | 1 | 10 | 0 |
| N46 | 0 | 10 | 80 |
| N46 | 0.1 | 10 | 100 |
| N46 | 0.3 | 10 | 0 |
| N46 | 0.5 | 10 | 0 |
| N46 | 0.7 | 10 | 0 |
| N46 | 1 | 10 | 0 |

Seed from the experiment shown in Table 6 were stored for one month, and then planted in flats to germinate. The germination results are shown in Table 8. Samples N46-1 through P38-12 are non-transgenic controls, no germination was observed after priming with Dormex®. Samples N46-13 through N46-21 are transgenic seed, N46-21 is a transgenic control primed with water, N46-13 and N46-14 are from a transgenic target line comprising PHP17797, which lacks the moCAH selectable marker, the remaining samples represent paired samples of seed from targeted linkage and activation of moCAH selectable marker from the donor, to the ubiquitin promoter in the target.

TABLE 8

| Corn Line | % Dormex | Lot | Total Seed | % Germination |
|---|---|---|---|---|
| N46-1 | 3 | 1 | 12 | 0 |
| N46-2 | 3 | 1 | 12 | 0 |
| N46-3 | 3 | 2 | 12 | 0 |
| N46-4 | 3 | 2 | 12 | 0 |
| N46-5 | 5 | 2 | 12 | 0 |
| N46-6 | 5 | 2 | 12 | 0 |
| P38-7 | 3 | 1 | 12 | 0 |
| P38-8 | 3 | 1 | 12 | 0 |
| P38-9 | 3 | 2 | 12 | 0 |
| P38-10 | 3 | 2 | 12 | 0 |
| P38-11 | 5 | 2 | 12 | 0 |
| P38-12 | 5 | 2 | 12 | 0 |
| N46-13 | 3 | 2 | 9 | 0 |
| N46-14 | 5 | 2 | 9 | 0 |
| P38-15 | 3 | 2 | 26 | 35 |
| P38-16 | 5 | 2 | 19 | 32 |
| N46-17 | 3 | 2 | 13 | 23 |
| N46-18 | 5 | 2 | 13 | 0 |
| N46-19 | 3 | 2 | 13 | 0 |
| N46-20 | 5 | 2 | 13 | 0 |
| N46-21 | 0 | — | 12 | 83 |

### E. Testing Priming Matrix Additives

A variety of additives can be included in the priming matrix, including surfactants, additional selective agents, fungicides, agents to modify osmotic potential, osmotic protectants, agents to extend storage shelf life, agents to enhance coating and/or perfusion, and the like.

1. Dormex®+DMSO

Non-transgenic maize seed from inbred N46 were primed with 0, 5, 10, 15, or 30% Dormex® solutions with and without 1% DMSO. For each treatment, 96 seeds were soaked in 50 ml of Dormexe solution in a petri dish for 3 hours at room temperature. After priming the seeds were transferred into pollination bags, and dried overnight in front of a fan. Dried primed seed were then planted into flats to germinate. Results are shown in Table 9.

TABLE 9

| Treatment | Total Seed | Seedlings | Germination % |
|---|---|---|---|
| Water | 96 | 92 | 96 |
| 5% Dormex ® | 96 | 0 | 0 |
| 10% Dormex ® | 96 | 0 | 0 |
| 15% Dormex ® | 92 | 0 | 0 |
| 30% Dormex ® | 96 | 0 | 0 |
| 1% DMSO | 95 | 88 | 93 |
| 5% Dormex ®, 1% DMSO | 96 | 0 | 0 |
| 10% Dormex ®, 1% DMSO | 96 | 0 | 0 |
| 15% Dormex ®, 1% DMSO | 96 | 0 | 0 |
| 30% Dormex ®, 1% DMSO | 96 | 0 | 0 |

2. Dormex®+Tween-20

Fifty F1 seeds from crosses of inbred donor lines, comprising PHP18000, to target lines, comprising PHP17797, were primed in 2.2% Dormexe +0.4% Tween-20 for 3 hours, then dried overnight before planting 25 seed in soil, with the remaining 25 primed seed germinated at 32° C. using germination paper. Linkage of the moCah gene from the donor to the ubi promoter in the target was confirmed by PCR, all germinated seeds were PCR positive for ubi::moCah linkage. Results are shown in Table 10.

TABLE 10

% Germination

| Corn Line | Soil | Paper |
|---|---|---|
| N46_3313853 | 72 | 84 |
| N46_3313686 | 44 | 80 |
| P38_3312969 | 4 | 8** |
| P38_3312982 | 0 | 1 |

**Germination very slow, at a later time point in the paper germination test 20% of seeds had germinated.

In another experiment, F1 seeds from crosses of N46 inbred donor lines, comprising PHP18000, to target lines, comprising PHP17797, were primed in 2.2% Dormex®+0.4% Tween-20 for 3 hours, then dried overnight before planting in soil. The germination results are shown in Table 11.

TABLE 11

| Corn Line | Total Seed | % Germination |
|---|---|---|
| N46_3313645 | 2 | 0 |
| N46_3313675 | 4 | 25 |
| N46_3313809 | 4 | 0 |
| N46_3313891 | 4 | 75 |
| N46_3313903 | 5 | 0 |
| N46_3313866 | 6 | 17 |
| N46_3313601 | 7 | 0 |
| N46_3313823 | 7 | 29 |
| N46_3313761 | 10 | 0 |
| N46_3313801 | 10 | 30 |
| N46_3313572 | 12 | 0 |
| N46_3313712 | 13 | 0 |
| N46_3313723 | 23 | 48 |
| N46_3313901 | 13 | 23 |
| N46_3313821 | 14 | 0 |
| TOTAL | 134 | 17 |

Priming with 2-10% v/v Dormex®+0.4% Tween-20, and paper germination at 25° C. or 32° C. was tested using non-transgenic N46 and P38 seed. Germination was checked at 3, 4, and/or 5 days after planting. Percent germination results are shown in Table 12.

TABLE 12

| | | | Paper Germination | | | |
|---|---|---|---|---|---|---|
| | % Dormex | Total | 25° C. | | 32° C. | |
| Corn Line | 0.4% Tw-20 | Seed | 4 day | 5 day | 3 day | 5 day |
| N46 | 2 | 20 | 0 | 0 | 0 | 0 |
| N46 | 2.2 | 20 | 0 | 0 | 0 | 0 |
| N46 | 2.5 | 20 | 0 | 0 | 0 | 0 |
| N46 | 3 | 20 | 0 | 0 | 0 | 0 |
| N46 | 5 | 20 | 0 | 0 | 0 | 0 |
| N46 | 10 | 20 | 0 | 0 | 0 | 0 |
| N46 | 0** | 20 | 80 | 80 | 85 | 90 |
| P38 | 2 | 20 | 0 | 0 | 0 | 0 |
| P38 | 2.2 | 20 | 0 | 0 | 0 | 0 |
| P38 | 2.5 | 20 | 0 | 0 | 0 | 0 |
| P38 | 3 | 20 | 0 | 0 | 0 | 0 |
| P38 | 0** | 20 | 95 | 95 | 100 | 100 |

**Water only treatment, no Tween-20 used.

### EXAMPLE 2

### Seed Priming with Glyphosate

Maize seeds are prepared by placing seed into a mesh bag. The seeds are then placed into a glyphosate solution to just cover seed. The seeds are incubated for varying periods of time in the dark at 20° C. Following the incubation, the seeds are removed from the glyphosate solution, rinsed with water, and allowed to dry for 1-3 days and then stored, or placed in flats to germinate.

A range of glyphosate concentrations, treatment times are tested on at least two corn inbreds. For example, glyphosate concentrations ranging from about 0.3 mM to 80 mM are used, which encompasses the concentration range used for spraying plants, with 18 mM (0.5%) being approximately 1× application rate. A range of soak times from about 10 minutes to about 5 hours or more is also tested, as are the conditions for drying and storage of primed seed.

Similar testing, as done in Examples 1-2, can be done to determine appropriate priming conditions for this and other selective agents in other corn inbred lines, corn hybrids, and other seeds such as soybean, rice, cotton, canola, etc.

### EXAMPLE 3

### Generating and Selecting a Targeted Seed

Any method can be used to provide a target site, a transfer cassette, and a recombinase to a plant to generate targeted seeds from any plant comprising a polynucleotide encoding a selectable marker operably linked to a promoter, which can be identified and selected by priming the whole population of seeds with the selective agent. Constructs are generated based on the method of transformation to be used. Further, any selective agent can be used, under a variety of conditions.

Events can further be characterized for copy number, homozygosity, gene expression, integrity of the inserted construct, and the like. Plants can be evaluated for any phenotypic effect due to the insertion of the target site, transfer cassette, recombinase, or other polynucleotides of interest. Further, plants can be crossed or outcrossed to generated homozygous lines, or transfer the component to another plant line.

### A. Transformation Vectors

Constructs comprising the transfer cassette or the target site are generated and used to transform plants to establish donor and acceptor plant lines, respectively. The target site and the transfer cassette are designed to allow for the selection of a targeted integration event by priming or soaking seed from putative targeted events with a selective agent. In this example, the target site and transfer cassette are designed to replace a FLP recombinase encoding sequence with a cyanamide hydratase encoding sequence, thereby operably linking and activating the selectable marker, and deactivating the recombinase. Optionally, standard techniques can be used to provide the recombinase, for example, providing a separate construct encoding the FLP recombinase operably linked to a promoter, placing the FLP coding region in a different location in the target construct, or placing the FLP coding region on the transfer cassette, or providing FLP DNA, mRNA, or protein transiently.

i. Agrobacterium vectors

Agrobacterium binary plasmids were made using the hybrid system described by Komari et al. ((1996) Plant J 10:165-174). Derivatives of pSB11 were built as intermediate T-DNA constructs containing the desired configuration between the T-DNA border sequences. Plasmid pSB11 was obtained from Japan Tobacco Inc. (Tokyo, Japan). Construction of pSB11 from pSB21, and construction of pSB21 from starting vectors, is described by Komari et al. ((1996) Plant J 10:165-174). Description of integration of the T-DNA plasmid into the superbinary plasmid pSB1 by homologous recombination can be found in EP672752 A1. The plasmid pSB1 was also obtained from Japan Tobacco Inc. These plasmids were used for Agrobacterium-mediated transformation after making the co-integrant in LBA4404. Electro-competent cells of the Agrobacterium strain LBA4404 harboring pSB1 were created using the protocol as described by Lin (1995) in Methods in Molecular Biology, ed. Nickoloff, J. A. (Humana Press, Totowa, N.J.). Cells and DNA were prepared for electroporation by mixing 1 μl plasmid DNA (˜100 ng) with 20 μl of competent cells in a Life Technologies (now Whatman Biometra) 0.15 cm electrode gap cuvette (Whatman Biometra #11608-031). Electroporation was performed in a Cell-Porator Electroporation device using the Pulse Control unit (Whatman Biometra #11604-014) at the 330 pF setting along with the Voltage Booster (Whatman Biometra #11612-017) set at 4 kW. The system delivers approximately 1.8 kV to the Agrobacterium cells. Successful recombination was verified by restriction analysis of the co-integrant plasmid following isolation and transformation back into E. coli DH5α cells for amplification.

PHP17797 target and FLPm expression vector comprises: RB—ubi pro::ubi intron/FRT1/ubi intron:: FLPm::pinII-CaMV35S enh::CaMV35S pro::adh1 intron::bar::pinII::FRT5-LB

The maize ubi intron is approximately 1010 nucleotides long. For construction of target site vector PHP17797, the 48-nucleotide FRT1 site was introduced, along with flanking restriction sites HindIII (5′) and BgIII (3′), between nucleotides 974 and 975 for a final approximate length of 1070 nucleotides. FRT site introduction can be done variety of ways, such as those described in U.S. Pat. No. 6,187,994. In PHP17797, the FRT5 site is in a short (65 nt) sequence between pinII and LB. Spectinomycin resistance is outside the T-DNA borders in the vector backbone. The final desired molecule was isolated from spectinomycin resistant E. coli isolates and verified by restriction digestion analysis.

PHP18000 moCAH transfer cassette vector comprises:

RB—FRT1/ubi intron::moCAH::pinII-35CaMV enh::ubi pro::ubi 5′ UTR::ubi intron::GFPm exon1::ST-LS1 intron::GFPm exon2::pinII::FRT5-LB PHP18000 comprises FRT1 linked to a truncated ubi intron such that it has the same sequence and configuration as the target (PHP17797), recombination with the target regenerates the ubi intron. The GFPm coding sequence contains the ST-LS1 intron from potato. Spectinomycin resistance is outside the T-DNA borders in the vector backbone. The final desired molecule was isolated from spectinomycin resistant E. coli isolates and verified by restriction digestion analysis.

### B. Generation of Donor and Acceptor Plants

Transgenic donor and acceptor plant lines can be established via any transformation method, for example Agrobacterium mediated infection or particle bombardment.

i. Agrobacterium Mediated Transformation

Agrobacterium mediated transformation of maize is performed essentially as described by Zhao (WO98/32326). Briefly, immature embryos are isolated from maize and the embryos contacted with a suspension of Agrobacterium containing a T-DNA, where the bacteria are capable of transferring the nucleotide sequence of interest to at least one cell of at least one of the immature embryos.

Step 1: Infection Step. In this step the immature embryos are immersed in an Agrobacterium suspension for the initiation of inoculation.
Step 2: Co-cultivation Step. The embryos are co-cultured for a time with the Agrobacterium.
Step 3: Resting Step. Optionally, following co-cultivation, a resting step may be performed. The immature embryos are cultured on solid medium with antibiotic, but without a selecting agent, for elimination of Agrobacterium and for a resting phase for the infected cells.
Step 4: Selection Step. Inoculated embryos are cultured on medium containing a selective agent and growing transformed callus is recovered. The immature embryos are cultured on solid medium with a selective agent resulting in the selective growth of transformed cells.
Step 5: Regeneration Step. Calli grown on selective medium are cultured on solid medium to regenerate the plants.

ii. Particle Bombardment of Maize

Immature maize embryos are bombarded with a DNA construct comprising the transfer cassette or comprising the target site. The recombinase can be provided on the target site construct, transfer cassette construct, or separately. Each of the constructs may also contain the selectable marker gene PAT (Wohlleben et al. (1988) Gene 70:25-37) that confers resistance to the herbicide Bialaphos. Transformation is performed as follows.

Preparation of Target Tissue: The ears are surface sterilized in 30% chlorox bleach plus 0.5% Micro detergent for 20 minutes, and rinsed two times with sterile water. The immature embryos are excised, placed embryo axis side down (scutellum side up), 25 embryos per plate, on 560Y medium for 4 hours and then aligned within the 2.5-cm target zone in preparation for bombardment.

Preparation of DNA: The DNA is precipitated onto 0.6 μm (average diameter) gold pellets using a CaCl₂ precipitation procedure as follows: 100 μl prepared gold particles in water; 10 μl (1 μg) DNA in TrisEDTA buffer (1 μg total); 100 μl 2.5 M CaCl₂; and, 10 μl 0.1 M spermidine.

Each reagent is added sequentially to the gold particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 μl 100% ethanol, and centrifuged for 30 seconds. After the liquid is removed, 105 μl 100% ethanol is added to the final gold particle pellet. For particle gun bombardment, the gold/DNA particles are briefly sonicated and 10 μl spotted onto the center of each macrocarrier and allowed to dry about 2 minutes before bombardment.

The sample plates of target embryos are bombarded using approximately 0.1 μg of DNA per shot using the Bio-Rad PDS-1000/He device (Bio-Rad Laboratories, Hercules, Calif.) with a rupture pressure of 650 PSI, a vacuum pressure of 27-28 inches of Hg, and a particle flight distance of 8.5 cm. Ten aliquots are taken from each tube of prepared particles/DNA.

Following bombardment, the embryos are kept on 560Y medium for 2 days, then transferred to 560R selection medium containing 3 mg/L Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. The acceptor plant will be monitored for phenotypic traits associated with both the site specific recombinase and the DNA construct comprising the target site, while the donor plant will be monitored for phenotypic traits associated with the DNA construct comprising the transfer cassette.

Medium 560Y comprises 4.0 g/L N6 basal salts (SIGMA C-1416), 1.0 ml/L Eriksson's Vitamin Mix (1000× SIGMA-1511), 0.5 mg/L thiamine HCl, 120 g/L sucrose, 1.0 mg/L 2,4-D, and 2.88 g/L L-proline (brought to volume with D-I H₂O following adjustment to pH 5.8 with KOH); 2.0 g/L Gelrite® (added after bringing to volume with D-I H₂O); and 8.5 mg/L silver nitrate (added after sterilizing the medium and cooling to room temperature).

Medium 560R comprises 4.0 g/L N6 basal salts (SIGMA C-1416), 1.0 ml/L Eriksson's Vitamin Mix (1000× SIGMA-1511), 0.5 mg/L thiamine HCl, 30.0 g/L sucrose, and 2.0 mg/L 2,4-D (brought to volume with D-I H₂O following adjustment to pH 5.8 with KOH); 3.0 g/L Gelrite (added after bringing to volume with D-I H₂O); and 0.85 mg/L silver nitrate and 3.0 mg/L bialaphos (both added after sterilizing the medium and cooling to room temperature).

Medium 288J comprises: 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl, and 0.40 g/L glycine brought to volume with D-I H₂O) (Murashige & Skoog (1962) Physiol Plant 15:473), 100 mg/L myo-inositol, 0.5 mg/L zeatin, 60 g/L sucrose, and 1.0 ml/L of 0.1 mM abscissic acid (brought to volume with D-I H₂O after adjusting to pH 5.6); 3.0 g/L Gelrite (added after bringing to volume with D-I H₂O); and 1.0 mg/L indoleacetic acid and 3.0 mg/L bialaphos (added after sterilizing the medium and cooling to 60° C).

Medium 272V comprises: 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl, and 0.40 g/L glycine brought to volume with D-I H₂O), 0.1 g/L myo-inositol, and 40.0 g/L sucrose (brought to volume with D-I H₂O after adjusting pH to 5.6); and 6 g/L bacto-agar (added after bringing to volume with D-I H₂O), sterilized and cooled to 60° C.

iii. Particle Bombardment of Soybean

A polynucleotide comprising a recombination site, transfer cassette, target site, other polynucleotide(s) of interest, selectable marker, and/or recombinase can be introduced into embryogenic suspension cultures of soybean by particle bombardment using essentially the methods described in Parrott et al. (1989) Plant Cell Rep 7:615-617. This method, with modifications, is described below.

Seed is removed from pods when the cotyledons are between 3 and 5 mm in length. The seeds are sterilized in a bleach solution (0.5%) for 15 minutes after which time the seeds are rinsed with sterile distilled water. The immature cotyledons are excised by first cutting away the portion of the seed that contains the embryo axis. The cotyledons are then removed from the seed coat by gently pushing the distal end of the seed with the blunt end of the scalpel blade. The cotyledons are then placed in petri dishes (flat side up) with SB1 initiation medium (MS salts, B5 vitamins, 20 mg/L 2,4-D, 31.5 g/L sucrose, 8 g/L TC Agar, pH 5.8). The petri plates are incubated in the light (16 hr day; 75-80 μE) at 26° C. After 4 weeks of incubation the cotyledons are transferred to fresh Sb1 medium. After an additional two weeks, globular stage somatic embryos that exhibit proliferative areas are excised and transferred to FN Lite liquid medium (Samoylov etal. (1998) In Vitro Cell Dev Biol Plant 34:8-13). About 10 to 12 small clusters of somatic embryos are placed in 250 ml flasks containing 35 ml of Sb172 medium. The soybean embryogenic suspension cultures are maintained in 35 mL liquid media on a rotary shaker, 150 rpm, at 26° C. with fluorescent lights (20 μE) on a 16:8 hour day/night schedule. Cultures are sub-cultured every two weeks by inoculating approximately 35 mg of tissue into 35 mL of liquid medium.

Soybean embryogenic suspension cultures are then transformed using particle gun bombardment (Klein et al. (1987) Nature 327:70; U.S. Pat. No. 4,945,050). A BioRad Biolisticä PDS1000/HE instrument can be used for these transformations. A selectable marker gene, which is used to facilitate soybean transformation, is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz etal. (1983) Gene 25:179-188) and the 3′ region of the nopaline synthase gene from the T-DNA of the Ti plasmid of Agrobacterium tumefaciens.

To 50 μL of a 60 mg/mL 1 μm gold particle suspension is added (in order): 5 μL DNA (1 μg/μL), 20 μl spermidine (0.1 M), and 50 μL CaCI2 (2.5 M). The particle preparation is agitated for three minutes, spun in a microfuge for 10 seconds and the supernatant removed. The DNA-coated particles are washed once in 400 μL 70% ethanol then resuspended in 40 μL of anhydrous ethanol. The DNA/particle suspension is sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. Membrane rupture pressure is set at 1100 psi and the chamber is evacuated to a vacuum of 28 inches mercury. The tissue is placed approximately 8 cm away from the retaining screen, and is bombarded three times. Following bombardment, the tissue is divided in half and placed back into 35 ml of FN Lite medium.

Five to seven days after bombardment, the liquid medium is exchanged with fresh medium. Eleven days post bombardment the medium is exchanged with fresh medium containing 50 mg/mL hygromycin. This selective medium is refreshed weekly. Seven to eight weeks post bombardment, green transformed tissue will be observed growing from untransformed, necrotic embryogenic clusters. Isolated green tissue is removed and inoculated into individual flasks to generate new, clonally propagated, transformed embryogenic suspension cultures. Each new line is treated as an independent transformation event. These suspensions are then subcultured and maintained as clusters of immature embryos, or tissue is regenerated into whole plants by maturation and germination of individual embryos.

### C. DNA Isolation from Callus and Leaf Tissues

Putative transformation events can be screened for the presence of the transgene. Genomic DNA is extracted from calli or leaves using a modification of the CTAB (cetyltriethylammonium bromide, Sigma H5882) method described by Stacey & Isaac (1994 In Methods in Molecular Biology Vol. 28, pp. 9-15, Ed. P. G. Isaac, Humana Press, Totowa, N.J.). Approximately 100-200 mg of frozen tissue is ground into powder in liquid nitrogen and homogenized in 1 ml of CTAB extraction buffer (2% CTAB, 0.02 M EDTA, 0.1 M TrisHCl pH 8,1.4 M NaCl, 25 mM DTT) for 30 min at 65° C. Homogenized samples are allowed to cool at room temperature for 15 min before a single protein extraction with approximately 1 ml 24:1 v/v chloroform:octanol is done. Samples are centrifuged for 7 min at 13,000 rpm and the upper layer of supernatant collected using wide-mouthed pipette tips. DNA is precipitated from the supernatant by incubation in 95% ethanol on ice for 1 h. DNA threads are spooled onto a glass hook, washed in 75% ethanol containing 0.2 M sodium acetate for 10 min, air-dried for 5 min and resuspended in TE buffer. Five μl RNAse A is added to the samples and incubated at 37° C. for 1 h. For quantification of genomic DNA, gel electrophoresis is performed using a 0.8% agarose gel in 1× TBE buffer. One microlitre of each of the samples is fractionated alongside 200, 400, 600 and 800 ng μl -1 λ uncut DNA markers.

### D. Providing the Transfer Cassette to the Acceptor Plant

A cross is performed between the donor maize plant having the transfer cassette, and the acceptor maize plant having the target site. Alternatively, any transformation method can be used to provide the transfer cassette by directly transformation into an acceptor line. Optionally, at least one of the maize plant lines is homozygous for the target site or transfer cassette. In addition, the acceptor plant genome may also have stably incorporated an expression cassette encoding the recombinase. The cross is performed between the donor and the acceptor plant. Seeds from the resulting cross are collected, and targeted integration events identified using seed priming to identify activation of the selectable marker from the transfer cassette.

### E. Identification of Targeted Seed

Maize seeds were prepared as described in Example 1. Targeted seeds have the transfer cassette integrated at the target site, activating moCAH and resistance to Dormex®. Non-targeted seed do not express moCAH and are susceptible to the Dormex® and cannot germinate. Table 4 in Example 1 shows results for targeted integration events in corn inbreds N46 and P38. Samples N46-1 through P38-12 are non-transgenic controls, no germination was observed after priming with Dormex®. Samples N46-13 through N46-21 are transgenic seed, N46-21 is a transgenic control primed with water, N46-13 and N46-14 are from a transgenic target line comprising PHP17797, which lacks the moCAH selectable marker, the remaining samples, P38-15 through N46-20, represent paired samples of seed from targeted linkage and activation of moCAH selectable marker from the donor, to the ubiquitin promoter in the target. Seeds that germinated were further analyzed for cyanamide hydratase enzyme activity, and PCR to confirm linkage of the ubi promoter to moCAH. Plants can be planted in the field or greenhouse for further analyses.

### EXAMPLE 4

### Activating and Screening for Two Markers

The seed priming method can be extended to screen for the presence, activation and/or expression of more than one selectable marker.

### A. Recombinase-Mediated Cassette Exchange Results in Activation of Two Marker Genes in the Target Locus.

i. Target A is constructed and introduced to produce Plants. The construct comprises:

Target A: Rb-Ubi Pro::Ubi intron::FRT1::FLP::pinII-CaMV35S::bar::pinII-TGA::FRT5::moCAH::pinII-Lb.

After introduction of the construct, transformed cells/tissues are selected on bialaphos-containing medium and regenerated to produce Target A plants. The expression of bar confers resistance to bialaphos. The tissue/plants can be further characterized, for example, DNA is extracted from regenerated T0 plants and subsequent Ti progeny to confirm that the above-introduced DNA is present as a single copy using standard Southern analysis methods. Integration of Target A produces plants having FLP recombinase activity and bialaphos resistance (FLP⁺, BLP^{r}). The moCAH sequence (3′ to the stop codon and FRT5) is not operably linked to the CaMV35S promoter, and therefore is not expressed in the Target A plants.

ii. Donor A is constructed and introduced to Target A plants by any method, including direct transformation, or by generating independent Donor A plant lines, and subsequently crossing the target and donor plants.
Donor A: Rb-CaMV35S Pro::bar::pinII-TGA-FRT1:GAT::pinII-Actin Pro::Actin intron::FRT5-Lb

If separate Donor A plants are generated, after introduction of the construct, transformed cells/tissues are selected on bialaphos-containing medium and regenerated to produce Donor A plants. The GAT sequence is not operably linked to a promoter, and therefore is not expressed in the donor plants.

iii. Activation of two markers by recombination via sexual crossing Target and donor plants are grown and crossed to each other. In the presence of the recombinase, the donor cassette, containing promoterless GAT and Actin Pro::Actin intron 5′ to FRT5, exchanges into the target locus, operably linking GAT to the ubiquitin promoter (Ubi Pro), and moCah to the Actin promoter (Actin Pro). The recombined product Target A′ locus results in expression of GAT and moCah and confers resistance to glyphosate (GLY^{r}) and cyanamide (CYA^{r}), respectively. Progeny seed from this cross can be primed with a combination of glyphosate and cyanamide. Optionally, progeny seed could be primed sequentially in two separate priming matrices comprising each separate herbicide, or the progeny seed could be primed with one herbicide, and the resistant plantlets could be leaf painted or sprayed with the second herbicide.

Progeny in which proper recombinase-mediated cassette exchange has occurred at both the FRT1 and FRT5 sites are FLP-, BLP^{r}, GLY^{r} and CYA^{r}. The recombination products Target A′ and Donor A′ are two independent loci and can be segregated away from each other in the next generation(s). Further analyses, such as PCR across the recombined FRT1 and FRT5 junctions, Southern analysis and/or sequencing can be used to further confirm that precise recombination mediated by FLP recombinase occurred during the cassette exchange.

### B. Recombinase-Mediated Cassette Exchange Results in Activation of Two Marker Genes in the Donor Locus.

i. Target B is constructed and introduced to produce plants. The construct comprises:

Target B: Rb-FRT1-moCah::pinII/Ubi Pro::Ubi intron::FLP::pinII-CaMV35S::bar::pinII-Actin Pro::Actin intron::FRT5-Lb.

After introduction of the construct, transformed cells/tissues are selected on bialaphos-containing medium and regenerated to produce Target B plants. The expression of bar confers resistance to bialaphos. The tissue/plants can be further characterized, for example, DNA is extracted from regenerated TO plants and subsequent Ti progeny to confirm that the above-introduced DNA is present as a single copy using standard Southern analysis methods. Integration of Target B produces plants having FLP recombinase activity and bialaphos resistance (FLP⁺, BLpr).

ii. Donor B is constructed and introduced to Target B plants by any method, including direct transformation, or by generating independent Donor B plant lines, and subsequently crossing the target and donor plants.

Donor B: Rb-CaMV35S Pro::bar::pinII-Ubi Pro::Ubi intron::FRT1::Ubi::GAT::pinII-TGA-FRT5:Actin Pro::YFP::pinIl-Lb

If separate Donor B plants are generated, after introduction of the construct, transformed cells/tissues are selected on bialaphos-containing medium and regenerated to produce Donor B plants.

iii. Activation of two markers by recombination via sexual crossing Target and donor plants are grown and crossed to each other. Donor B contains an active Ubi::GAT:pinII which will be inserted into the target locus by recombinase-mediated cassette exchange. In F1 progeny, the desired target product cannot be identified by screening for GAT, since GAT is operably linked to a promoter before and after recombination. However, in the process of cassette exchange, the inactive moCah and YFP from the Target B locus are operably linked to promoters in the recombined Donor B′ locus, and can be used as an indication that cassette exchange occurred. Progeny that are CYA^{r} and YFP⁺ (leaves can be measured with a hand-held OS1-FL meter; Opti-Sciences, Inc., 164 Westford Rd., Tyngsboro, Mass. 01879) indicate proper cassette exchange between the two loci. These CYA^{r}, YFP⁺ plants can be outcrossed to wild-type plants, wherein the CYA^{r}, YFP⁺ traits segregate as a single unit in the progeny with Donor B′ while the GLY^{r} segregates away from CYA^{r}, YFP⁺. Putative Target B′ seed can be initially screened by seed priming in a glyphosate solution, and then further analyzed. For example, the recombined Target B′ locus will further be bar⁻ (BLP^{s}) and FLP⁻ due to the exchange of these cassettes into the Donor locus. Bialophos sensitivity can be assayed by leaf painting, and/or PCR analyses across recombined FRT1 and FRT5 junctions and/or bar cassette or other genes, as well as Southern analysis and sequencing will be used to confirm that precise recombination mediated by FLP recombinase occurred during the cassette exchange.

### C. Recombinase-Mediated Cassette Exchange Using Two Independent Recombination Systems.

i. Target C is constructed and introduced to produce plants. The construct comprises:

### Target C: Rb-Ubi Pro::FRT1::FLP::pinII-(CaMV35Spro::bar::CaMV35S term)-GZ3′-pinII::Cre::loxP::Actin Pro-Lb

After introduction of the construct, transformed cells/tissues are selected on bialaphos-containing medium and regenerated to produce Target C plants. Expression of bar confers resistance to bialaphos. The tissue/plants can be further characterized, for example, DNA is extracted from regenerated T0 plants and subsequent T1 progeny to confirm that the above-introduced DNA is present as a single copy using standard Southern analysis methods. Target C plants are FLP⁺, Cre⁺, BLP^{r}.

ii. Donor C is constructed and introduced to Target C plants by any method, including direct transformation, or by generating independent Donor C plant lines, and subsequently crossing the target and donor plants.

### Donor C: Rb-(CaMV35S Pro::bar::pinII)::FRT1::GAT::pinII-GZ3′-pinII::moCah::loxP-Lb

If separate Donor C plants are generated, after introduction of the construct, transformed cells/tissues are selected on bialaphos-containing medium and regenerated to produce Donor C plants, which are bialaphos resistant (BLP^{r}). GAT and moCah sequences are promoterless, and not expressed in the donor plants.

iii. Activation of two markers by recombination via sexual crossing Target and donor plants are grown and crossed to each other. In the presence of the recombinases Donor C, containing inactive GAT and moCah sequences, is exchanged into the Target C locus operably linking GAT to the ubiquitin promoter, and moCah to the actin promoter, to generate Target C′ conferring resistance to the herbicides glyphosate (GLY^{r}) and cyanamide (CYA^{r}), respectively. Progeny seed comprising Target C′ can be identified by seed priming with both herbicides, either simultaneously or sequentially. Optionally, seed priming is done with only one herbicide, and the resulting seedlings can be leaf painted or sprayed with the second herbicide. The recombined loci, Target C′ and Donor C′ are independent and can be segregated away from each other in the next generation. Further characterization, such as PCR analyses for genes, across FRT1 and loxP junctions, as well as Southern analysis and sequencing can be used to confirm that precise recombinase-mediated exchange occurred.

### D. Recombinase-Mediated Cassette Exchange Results in Activation of One Marker Gene in the Target Locus and One Marker Gene in the Donor Locus.

These methods and compositions can also be done in other crops, such as soybean. For example, Jack, a Glycine max (I.) Merrill cultivar can be transformed using particle bombardment.

i. Target D is constructed and introduced to produce plants. The construct comprises:

Target D: SCP1 pro::FRT1::FLP::pinII-CaMV35S pro::HYG::nos term::Kti3 pro-FRT6

After introduction of the construct, transformed cells/tissues are selected on hygromycin-containing medium and regenerated to produce Target D plants. The tissue/plants can be further characterized, for example, DNA is extracted from 15 regenerated T0 plants and subsequent T1 progeny to confirm that the above-introduced DNA is present as a single copy using standard Southern analysis methods. Target D plants are FLP⁺, HYG^{r}.

ii. Donor D is constructed and introduced to Target D plants by any method,

including direct transformation, or by generating independent Donor D plant lines, and subsequently crossing the target and donor plants.

Donor D: CaMV35S term-TGA-FRT1::Gm-Als::Gm-Als term-CaMV35S pro::GUS::nos term-TGA-FRT6::AmCyan1::KTI3 term

iii. Activation of two markers by recombination via transformation Target D plants (FLP⁺, HYG^{r}) are transformed with Donor D construct using particle bombardment.

Recombinase-mediated exchange will produce recombined Target D′ (containing SCP1 pro::FRT1::Gm-Als::Gm-Als term-CaMV35S pro::GUS::nos term-TGA::FRT6), and recombined Donor D′ (containing CaMV35S term-TGA-FRT1::FLP::pinII term-CaMV35S pro::HYG::nos term::Kti3 pro::FRT6::AmCyan1::Kti3 term).

After introduction of Donor D, cells/tissues can placed on media containing chlorsulfuron. Alternatively, cells/tissues can be placed on media to generate plants and seeds. The seeds produced can be screened by seed priming with a matrix comprising chlorsulfuron. Expression of Gm-ALS confers resistance to herbicides that inhibit acetolactate synthase (ALS), for example sulfonylurea herbicides such as chlorsulfuron. Cells/seeds containing Target D′ will be selected for by resistance to chlorsulfuron, and can be further screened for GUS, for example, histochemically (Jefferson, et al. (1987) EMBO J 6:3901-3907). The recombined Donor D′ is AmCyan1+due to linkage to KTI pro, and can be identified by screening for blue fluorescence.

### E. Recombinase-Mediated Cassette Exchange via Direct Delivery of the Donor for Activation of Two Marker Genes in the Target Locus.

i. Target E is constructed and introduced to produce plants. The construct comprises:

Target E: Rb-Ubi pro::FRT1::YFP::pinII term-Ubi pro::luciferase::pinII-GZ3′-term-IN2-1 term::bar::FRT5::Actin pro-Lb.

After introducing Target E, cells/tissues are placed on selection media containing bialophos. Transformed cells are YFP⁺, BLP^{r}, Luc⁺. Cells growing on selection media that are also YFP⁺ are regenerated as target plants.

ii. Donor E is constructed and introduced to Target E plants by any method, including direct transformation, or by generating independent Donor E plant lines, and subsequently crossing the target and donor plants.

Target E plants can be grown and selfed, or crossed to non-transgenic plants, and immature embryos isolated. Embryos expressing YFP are transformed using particle bombardment. Two plasmids are used, Donor E, and Plasmid R which comprises the recombinase:

Donor E: Rb-TGA-FRT1::AmCyan::pinII term-Ubi pro::moCAH::pinII term-GZ3′-IN2-1 term: :GAT: :FRT5-TGA-Lb.
Plasmid R: Ubi pro::FLP::pinII term.
Plasmid R is typically used at a lower DNA concentration for transient expression, rather than stable integration into the genome. If Plasmid R integrates, it can be removed through outcrossing and segregation of the Plasmid R locus from the Target E or E′ locus.

iii. Activation of two markers by recombination via transformation After bombardment with Donor E and Plasmid R, the cells/tissues can placed on selection media containing glyphosate. Alternatively, cells/tissues can be grown on non-selective media to generate plants and seeds. At any time, Target E′ can be screened for by looking for AmCyan⁺. Progeny seed comprising Target E′ can be identified by seed priming with both selective agents, either simultaneously or sequentially. Optionally, seed priming is done with only one selective agent, and the resulting seedlings can be leaf painted or sprayed with the second selective agent. Seeds/cultures/plants expressing all three genes can be further characterized, for example by PCR, to confirm that recombination occurred at the FRT1 and FRT5 junctions.

### F. Recombinase-Mediated Integration Results in Activation of Two Marker Genes in the Target Locus.

### Target sites comprising two promoters operably linked to one recombination site can be used.

i. Target F is constructed and introduced to produce plants. The construct comprises:

Target F: Rb-CaMV35S pro::bar::pinII term-Ubi pro::FRT1::Actin pro (3′-5′ orientation)-Lb.
After introducing Target F, cells/tissues are placed on selection media containing bialophos. Transformed cells are BLP^{r} and are regenerated as target plants.

ii. Donor F is constructed and introduced to Target F plants as a circular construct:

Donor F:—pinII term::moCAH::FRT1::GAT::term—
The recombinase is provided by co-transformation with a separate construct comprising:
Plasmid R: Ubi pro::FLP::pinII term.
Plasmid R is typically used at a lower DNA concentration for transient expression, rather than stable integration into the genome. If Plasmid R integrates, it can be removed through outcrossing and segregation of the Plasmid R locus from the Target F or F′ locus.

iii. Activation of two markers by recombination via transformation After bombardment with Donor F and Plasmid R, the cells/tissues can placed on selection media containing glyphosate. Alternatively, cells/tissues can be grown on non-selective media to generate plants and seeds. Progeny seed comprising Target F′ can be identified by seed priming with both herbicides, either simultaneously or sequentially. Optionally, seed priming is done with only one herbicide, and the resulting seedlings can be leaf painted or sprayed with the second herbicide. Seeds/cultures/plants can be further characterized, for example by PCR, to confirm the recombination junctions.

### G. Recombinase-Mediated Integration Results in Activation of Two Marker Genes in the Target Locus.

### Target sites comprising two promoters operably linked to one recombination site can be used.

i. Target G is constructed and introduced to produce plants. The construct comprises:

Target G: Rb-Ubi pro::FRT1::bar::pinII term::Actin pro (3′-5′ orientation)-Lb.
After introducing Target F, cells/tissues are placed on selection media containing bialophos. Transformed cells are BLP^{r} and are regenerated as target plants.

ii. Donor G is constructed and introduced to Target G plants by generating independent Donor G plant lines using any transformation method, and subsequently crossing the target and donor plants. In the presence of FLP recombinase, Donor G will be excised and circularized, and can then recombine and integrate at the Target G site. Optionally, Donor G can be constructed as a replicon, such as a viral replicon, wherein after excision and circularization, the donor will be replicated to generate higher copy number. The recombinase can be provided in either the target line or the donor line, and segregated away from the recombined Target G′ product.

Donor G: Rb—ubi pro::bar::pinII term—FRT1::GAT::term—pinII term::moCAH::FRT1—Lb
After introducing Donor G, cells/tissues are placed on selection media containing bialophos. BLP^{r} events are regenerated into Donor G plants and used for crossing.

iii. Activation of two markers by recombination via crossing In the presence of FLP recombinase, Donor G will be excised and circularized, and can then recombine and integrate at the Target G site. Optionally, Donor G can be constructed as a replicon, such as a viral replicon, wherein after excision and circularization, the donor will be replicated to generate higher copy number. The recombinase can be provided in either the target line or the donor line, and segregated away from the recombined Target G′ product. Optionally, the polynucleotide encoding FLP is linked to an inducible or tissue-specific promoter, in order to control FLP availability, and possibly minimize the reverse reaction. Progeny seed comprising Target G′ can be identified by seed priming with both herbicides, either simultaneously or sequentially. Optionally, seed priming is done with only one herbicide, and the resulting seedlings can be leaf painted or sprayed with the second herbicide. Seeds/cultures/plants can be further characterized, for example by PCR, to confirm the recombination junctions.

The articles “a” and “an” refer to one or more than one of the grammatical object of the article. By way of example, “an element” means one or more element.

All book, journal, patent publications and grants mentioned in the specification are indicative of the level of those skilled in the art. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

What is claimed is:

1. A method to identify a plant having a DNA construct stably incorporated in its genome comprising:
(a) providing a population of seeds, wherein at least one seed in the population has stably incorporated into its genome the DNA construct comprising the following operably linked components in the following order: a promoter active in the seed, a first recombination site, a polynucleotide encoding a first selection marker that confers resistance to a selective agent, and a second recombination site;
(b) contacting the population of seeds with a priming matrix comprising an effective concentration of the selection agent for a time sufficient to produce a population of primed seeds; and,
(c) incubating the population of primed seed under germination conditions, thereby identifying plants having the DNA construct stably incorporated into their genome.

2. The method of claim 1, wherein the first and the second recombination sites are dissimilar and non-recombinogenic with each other.

3. The method of claim 1, wherein the selective agent comprises an herbicide, a growth regulator, or an antibiotic.

4. The method of claim 3, wherein the selective agent is selected from the group consisting of glufosinate, phosphinothricin, glyphosate, bromoxynil, methotrexate, imidazolinones, sulfonylureas, cyanamide, kanamycin, G418, neomycin, and hygromycin B.

5. The method of claim 3, wherein the first selection marker is selected from the group consisting of Bar, phosphinothricin acetyltransferase (PAT), glyphosate oxidoreductase (GOX), 5-enolpyruvylshikimate-3-phosphate synthase (EPSPS), CP4, glyphosate N-acetyltransferase (GAT), bromoxynil nitrilase (BXN), dihydrofolate reductase, acetolactate synthase (ALS), cyanamide hydratase (CAH), neomycin phosphotransferase (nptil), aminoglycoside 3′-phosphotransferase (APH3′ II), and hygromycin phosphotransferase (hph).

6. The method of claim 5, wherein the polynucleotide encoding the first selection marker has been synthesized using maize preferred codons.

7. The method of claim 1, wherein the DNA construct comprises in the following order: a first expression unit comprising the promoter active in the seed operably linked to the first recombination site operably linked to the polynucleotide encoding the first selection marker; and a second expression unit comprising in the following order, a second promoter active in the plant operably linked to a polynucleotide of interest, and the second recombination site.

8. The method of claim 7, wherein the polynucleotide of interest encodes a second selection marker, wherein the second selection marker is distinct from the first selection marker by having a different selection means.

9. The method of claim 1, wherein the plant further has stably incorporated into its genome a polynucleotide encoding a site specific recombinase.

10. The method of claim 9, wherein the site specific recombinase comprises a FLP recombinase, a Cre recombinase, a lambda integrase, a SSV1 integrase, or a phiC31 integrase.

11. The method of claim 1, wherein at least one of the first or the second recombination sites are selected from the group consisting of a lox site, a mutant lox site, a FRT site, a mutant FRT site, an att site, and a mutant att site.

12. The method of claim 1, wherein the plant is maize, wheat, rice, sorghum, rye, oat, barley, millet, Brassica, alfalfa, sunflower, safflower, soybean, tobacco, cotton, or Arabidopsis.

13. A method of priming a seed comprising
(a) providing a seed having stably incorporated into its genome a DNA construct comprising, in the following order, a promoter active in the seed operably linked to a first recombination site operably linked to a polynucleotide encoding a selection marker that confers resistance to a selective agent; and
(b) contacting the seed with a priming matrix comprising an effective concentration of the selective agent for a time sufficient to produce a primed seed.

14. The method of claim 13, wherein the DNA construct comprises in the following order the promoter operably linked to the first recombination site operably linked the polynucleotide encoding the selection marker, and a second recombination site.

15. The method of claim 14, wherein the first and the second recombination sites are dissimilar and non-recombinogenic with each other.

16. A composition comprising a seed having stably incorporated into its genome a DNA construct comprising, in the following order, a promoter active in the seed operably linked to a first recombination site operably linked to a polynucleotide encoding a selection marker that confers resistance to a selective agent; and, a priming matrix comprising an effective concentration of the selective agent.

17. The composition of claim 16, wherein the DNA construct comprises in the following order, the promoter operably linked to the first recombination site operably linked to the polynucleotide encoding the selection marker, and a second recombination site.

18. The composition of claim 17, wherein the first and the second recombination sites are dissimilar and non-recombinogenic with each other.

19. The composition claim 16, wherein the DNA construct further comprises a polynucleotide of interest.

20. The composition of any one of claim 16, wherein the DNA construct further comprises a second selection marker.

21. The composition of claim 20, wherein the second selection marker confers resistance to a second selective agent.

22. The composition of claim 20, wherein the priming matrix further comprises the second selective agent.

23. The composition of claim 16, wherein the seed is from a plant selected from the group consisting of maize, wheat, rice, sorghum, rye, oat, barley, millet, Brassica, alfalfa, sunflower, safflower, soybean, tobacco, cotton, and Arabidopsis.
